SEQUENCE LISTING (24 sequences)

SEQ ID NO: 1 - DNA, 2210 bp, Unknown
  CDS (224)...(1951): Fungal isolate from soil sample
acgcggggtgcccacggacaacaattcccttaggattatctcctgtattgaatacactctactttgcaactttacctattattcgactttcttttagaggagcagcattgtcatcattacctgcccctccatctgatacctaccttacattgtcgccaacacacctataagccataatataccgactcaaagcaaaccacgcccattgtttgattgtttaatcatggccagcatcaacatcagggtgcagaatctcgagcaacccatggacgttgccgagtatctttttcggcgtctccacgaaatcggcattcgctccatccacggtcttccaggcgattacaaccttcttgccctcgactatttgccatcatgtggcctgagatgggttggcagcgtcaacgaactcaatgctgcttatgctgctgatggctatgcccgcgtcaagcagatgggagctctcatcaccacttttggagtgggagagctctcagccatcaatggcgttgccggtgccttttcggaacacgtcccagtcgttcacattgttggctgcccttccactgtctcgcagcgaaacggcatgctcctccaccacacgcttggaaacggcgacttcaacatctttgccaacatgagcgctcaaatctcttgcgaagtggccaagctcaccaaccctgccgaaattgcgacccagatcgaccatgccctccgcgtttgcttcattcgttctcggcccgtctacatcatgcttcccaccgatatggtccaggccaaagtagaaggtgccagactcaaggaaccaattgacttgtcggagcctccaaatgatcccgagaaagaagcatacgtcgttgacgttgtcctcaagtayctccgtgctgcaaagaaccccgtcatccttgtcgatgcttgtgctatccgtcatcgtgttcttgatgaggttcatgatctcatcgaaaagacaaacctccctgtctttgtcactcctatgggcaaaggtgctgttaacgaagaacacccgacatatggtggtgtctatgccggtgacggctcacatccgcctcaagttaaggacatggttgagtcttctgatttgatattgacaatcggtgctctcaagagcgacttcaacactgctggcttctcttaccgtacctcacagctgaacacgattgatctacacagcgaccactgcattgtcaaatactcgacatatccaggtgtccagatgaggggtgtgctgcgacaagtgattaagcagctcgatgcatctgagatcaacgctcagccagcgccagtcgtcgagaatgaagttgccaaaaaccgagataactcacccgtcattacacaagctttcttctggccgcgcgtgggagagttcctgaagaagaacgacatcgtcattaccgagactggaacagccaactttggcatctgggatactaagtttccctctggcgttactgcgctttctcaggtcctttggggaagcattggttggtccgttggtgcctgccaaggagccgttcttgcagccgccgatgacaacagcgatcgcagaactatcctctttgttggtgatggctcattccagctcactgctcaagaattgagcacaatgattcgtctcaagctgaagcccatcatctttgtcatctgcaacgatggctttaccattgaacgattcattcacggcatggaagccgagtacaacgacatcgcaaattgggacttcaaggctctggttgacgtctttggcggctctaagacggccaagaagttcgccgtcaagaccaaggacgagctggacagccttctcacagaccctacctttaacgccgcagaatgcctccagtttgtcgagctatatatgcccaaagaagatgctcctcgagcattgatcatgactgcagaagctagcgcgaggaacaatgccaagacagagtaaagtggactgtcatgaaggccgatttaccacctcataaattgtaatagacctgatacacatagatcaaggcaggtaccgatcattaatcaagcaggtttggatggggaaggattttgaaaatgaggaaacgatgggatgatatttggaataactggccattattttgagtacttataaacaaatttgaagttcaattttttttcaaaaaaaaaaaaaaaaaaaaaaaaaaaaaaaaaaaaaaaaaaaaaaaaaaaaaaaa

SEQ ID NO: 2 - DNA, 1725 bp, Unknown
  CDS (1)...(1725): Fungal isolate from soil sample
atggccagcatcaacatcagggtgcagaatctcgagcaacccatggacgttgccgagtatctttttcggcgtctccacgaaatcggcattcgctccatccacggtcttccaggcgattacaaccttcttgccctcgactatttgccatcatgtggcctgagatgggttggcagcgtcaacgaactcaatgctgcttatgctgctgatggctatgcccgcgtcaagcagatgggagctctcatcaccacttttggagtgggagagctctcagccatcaatggcgttgccggtgccttttcggaacacgtcccagtcgttcacattgttggctgcccttccactgtctcgcagcgaaacggcatgctcctccaccacacgcttggaaacggcgacttcaacatctttgccaacatgagcgctcaaatctcttgcgaagtggccaagctcaccaaccctgccgaaattgcgacccagatcgaccatgccctccgcgtttgcttcattcgttctcggcccgtctacatcatgcttcccaccgatatggtccaggccaaagtagaaggtgccagactcaaggaaccaattgacttgtcggagcctccaaatgatcccgagaaagaagcatacgtcgttgacgttgtcctcaagtayctccgtgctgcaaagaaccccgtcatccttgtcgatgcttgtgctatccgtcatcgtgttcttgatgaggttcatgatctcatcgaaaagacaaacctccctgtctttgtcactcctatgggcaaaggtgctgttaacgaagaacacccgacatatggtggtgtctatgccggtgacggctcacatccgcctcaagttaaggacatggttgagtcttctgatttgatattgacaatcggtgctctcaagagcgacttcaacactgctggcttctcttaccgtacctcacagctgaacacgattgatctacacagcgaccactgcattgtcaaatactcgacatatccaggtgtccagatgaggggtgtgctgcgacaagtgattaagcagctcgatgcatctgagatcaacgctcagccagcgccagtcgtcgagaatgaagttgccaaaaaccgagataactcacccgtcattacacaagctttcttctggccgcgcgtgggagagttcctgaagaagaacgacatcgtcattaccgagactggaacagccaactttggcatctgggatactaagtttccctctggcgttactgcgctttctcaggtcctttggggaagcattggttggtccgttggtgcctgccaaggagccgttcttgcagccgccgatgacaacagcgatcgcagaactatcctctttgttggtgatggctcattccagctcactgctcaagaattgagcacaatgattcgtctcaagctgaagcccatcatctttgtcatctgcaacgatggctttaccattgaacgattcattcacggcatggaagccgagtacaacgacatcgcaaattgggacttcaaggctctggttgacgtctttggcggctctaagacggccaagaagttcgccgtcaagaccaaggacgagctggacagccttctcacagaccctacctttaacgccgcagaatgcctccagtttgtcgagctatatatgcccaaagaagatgctcctcgagcattgatcatgactgcagaagctagcgcgaggaacaatgccaagacagag

SEQ ID NO: 3 - protein, 575 aa, Unknown
  Fungal isolate from soil sample
MASINIRVQNLEQPMDVAEYLFRRLHEIGIRSIHGLPGDYNLLALDYLPSCGLRWVGSVNELNAAYAADGYARVKQMGALITTFGVGELSAINGVAGAFSEHVPVVHIVGCPSTVSQRNGMLLHHTLGNGDFNIFANMSAQISCEVAKLTNPAEIATQIDHALRVCFIRSRPVYIMLPTDMVQAKVEGARLKEPIDLSEPPNDPEKEAYVVDVVLKYLRAAKNPVILVDACAIRHRVLDEVHDLIEKTNLPVFVTPMGKGAVNEEHPTYGGVYAGDGSHPPQVKDMVESSDLILTIGALKSDFNTAGFSYRTSQLNTIDLHSDHCIVKYSTYPGVQMRGVLRQVIKQLDASEINAQPAPVVENEVAKNRDNSPVITQAFFWPRVGEFLKKNDIVITETGTANFGIWDTKFPSGVTALSQVLWGSIGWSVGACQGAVLAAADDNSDRRTILFVGDGSFQLTAQELSTMIRLKLKPIIFVICNDGFTIERFIHGMEAEYNDIANWDFKALVDVFGGSKTAKKFAVKTKDELDSLLTDPTFNAAECLQFVELYMPKEDAPRALIMTAEASARNNAKTE

SEQ ID NO: 4 - DNA, 835 bp, Unknown
  CDS (3)...(596): Fungal isolate from soil sample
ctttcttctggccgcgcgtgggagagttcctgaagaagaacgacatcgtcattaccgagactggaacagccaactttggcatctgggatactaagtttccctctggcgttactgcgctttctcaggtcctttggggaagcattggttggtccgttggtgcctgccaaggagccgttcttgcagccgccgatgacaacagcgatcgcagaactatcctctttgttggtgatggctcattccagctcactgctcaagaattgagcacaatgattcgtctcaagctgaagcccatcatctttgtcatctgcaacgatggctttaccattgaacgattcattcacggcatggaagccgagtacaacgacatcgcaaattgggacttcaaggctctggttgacgtctttggcggctctaagacggccaagaagttcgccgtcaagaccaaggacgagctggacagccttctcacagaccctacctttaacgccgcagaatgcctccagtttgtcgagctatatatgcccaaagaagatgctcctcgagcattgatcatgactgcagaagctagcgcgaggaacaatgccaagacagagtaaagtggactgtcatgaaggccgatttaccacctcataaattgtaatagacctgatacacatagatcaaggcaggtaccgatcattaatcaagcaggtttggatggggaaggattttgaaaatgaggaaacgatgggatgatatttggaataactggccattattttgagtacttataaacaaatttgaagttcaattttttttcaaaaaaaaaaaaaaaaaaaaaaaaaaaaaaaaaaaa

SEQ ID NO: 5 - DNA, 591 bp, Unknown
  CDS (1)...(591): Fungal isolate from soil sample
ttcttctggccgcgcgtgggagagttcctgaagaagaacgacatcgtcattaccgagactggaacagccaactttggcatctgggatactaagtttccctctggcgttactgcgctttctcaggtcctttggggaagcattggttggtccgttggtgcctgccaaggagccgttcttgcagccgccgatgacaacagcgatcgcagaactatcctctttgttggtgatggctcattccagctcactgctcaagaattgagcacaatgattcgtctcaagctgaagcccatcatctttgtcatctgcaacgatggctttaccattgaacgattcattcacggcatggaagccgagtacaacgacatcgcaaattgggacttcaaggctctggttgacgtctttggcggctctaagacggccaagaagttcgccgtcaagaccaaggacgagctggacagccttctcacagaccctacctttaacgccgcagaatgcctccagtttgtcgagctatatatgcccaaagaagatgctcctcgagcattgatcatgactgcagaagctagcgcgaggaacaatgccaagacagag

SEQ ID NO: 6 - protein, 197 aa, Unknown
  Fungal isolate from soil sample
FFWPRVGEFLKKNDIVITETGTANFGIWDTKFPSGVTALSQVLWGSIGWSVGACQGAVLAAADDNSDRRTILFVGDGSFQLTAQELSTMIRLKLKPIIFVICNDGFTIERFIHGMEAEYNDIANWDFKALVDVFGGSKTAKKFAVKTKDELDSLLTDPTFNAAECLQFVELYMPKEDAPRALIMTAEASARNNAKTE

SEQ ID NO: 7 - DNA, 678 bp, Unknown
  CDS (1)...(678): Fungal isolate from soil sample
acatatccaggtgtccagatgaggggtgtgctgcgacaagtgattaagcagctcgatgcatctgagatcaacgctcagccagcgccagtcgtcgagaatgaagttgccaaaaaccgagataactcacccgtcattacacaagctttcttctggccgcgcgtgggagagttcctgaagaagaacgacatcgtcattaccgagactggaacagccaactttggcatctgggatactaagtttccctctggcgttactgcgctttctcaggtcctttggggaagcattggttggtccgttggtgcctgccaaggagccgttcttgcagccgccgatgacaacagcgatcgcagaactatcctctttgttggtgatggctcattccagctcactgctcaagaattgagcacaatgattcgtctcaagctgaagcccatcatctttgtcatctgcaacgatggctttaccattgaacgattcattcacggcatggaagccgagtacaacgacatcgcaaattgggacttcaaggctctggttgacgtctttggcggctctaagacggccaagaagttcgccgtcaagaccaaggacgagctggacagccttctcacagaccctacctttaacgccgcagaatgcctccagtttgtcgagctatatatgcccaaagaagatgct

SEQ ID NO: 8 - protein, 226 aa, Unknown
  Fungal isolate from soil sample
TYPGVQMRGVLRQVIKQLDASEINAQPAPVVENEVAKNRDNSPVITQAFFWPRVGEFLKKNDIVITETGTANFGIWDTKFPSGVTALSQVLWGSIGWSVGACQGAVLAAADDNSDRRTILFVGDGSFQLTAQELSTMIRLKLKPIIFVICNDGFTIERFIHGMEAEYNDIANWDFKALVDVFGGSKTAKKFAVKTKDELDSLLTDPTFNAAECLQFVELYMPKEDA

SEQ ID NO: 9 - DNA, 1636 bp, Unknown
  CDS (1)...(1377): Fungal isolate from soil sample
cgaaacggcatgctcctccaccacacgcttggaaacggcgacttcaacatctttgccaacatgagcgctcaaatctcttgcgaagtggccaagctcaccaaccctgccgaaattgcgacccagatcgaccatgccctccgcgtttgcttcattcgttctcggcccgtctacatcatgcttcccaccgatatggtccaggccaaagtagaaggtgccagactcaaggaaccaattgacttgtcggagcctccaaatgatcccgagaaagaagcatacgtcgttgacgttgtcctcaagtacctccgtgctgcaaagaaccccgtcatccttgtcgatgcttgtgctatccgtcatcgtgttcttgatgaggttcatgatctcatcgaaaagacaaacctccctgtctttgtcactcctatgggcaaaggtgctgttaacgaagaacacccgacatatggtggtgtctatgccggtgacggctcacatccgcctcaagttaaggacatggttgagtcttctgatttgatattgacaatcggtgctctcaagagcgacttcaacactgctggcttctcttaccgtacctcacagctgaacacgattgatctacacagcgaccactgcattgtcaaatactcgacatatccaggtgtccagatgaggggtgtgctgcgacaagtgattaagcagctcgatgcatctgagatcaacgctcagccagcgccagtcgtcgagaatgaagttgccaaaaaccgagataactcacccgtcattacacaagctttcttctggccgcgcgtgggagagttcctgaagaagaacgacatcgtcattaccgagactggaacagccaactttggcatctgggatactaagtttccctctggcgttactgcgctttctcaggtcctttggggaagcattggttggtccgttggtgcctgccaaggagccgttcttgcagccgccgatgacaacagcgatcgcagaactatcctctttgttggtgatggctcattccagctcactgctcaagaattgagcacaatgattcgtctcaagctgaagcccatcatctttgtcatctgcaacgatggctttaccattgaacgattcattcacggcatggaagccgagtacaacgacatcgcaaattgggacttcaaggctctggttgacgtctttggcggctctaagacggccaagaagttcgccgtcaagaccaaggacgagctggacagccttctcacagaccctacctttaacgccgcagaatgcctccagtttgtcgagctatatatgcccaaagaagatgctcctcgagcattgatcatgactgcagaagctagcgcgaggaacaatgccaagacagagtaaagtggactgtcatgaaggccgatttaccacctcataaattgtaatagacctgatacacatagatcaaggcaggtaccgatcattaatcaagcaggtttggatggggaaggattttgaaaatgaggaaacgatgggatgatatttggaataactggccattattttgagtacttataaacaaatttgaagttcaattttttttcaaaaaaaaaaaaaaaaaaaaaaaaaaaaaaaaaaaaaaaaaaaaaaaaaaaaaaaa

SEQ ID NO: 10 - DNA, 1374 bp, Unknown
  CDS (1)...(1374): Fungal isolate from soil sample
cgaaacggcatgctcctccaccacacgcttggaaacggcgacttcaacatctttgccaacatgagcgctcaaatctcttgcgaagtggccaagctcaccaaccctgccgaaattgcgacccagatcgaccatgccctccgcgtttgcttcattcgttctcggcccgtctacatcatgcttcccaccgatatggtccaggccaaagtagaaggtgccagactcaaggaaccaattgacttgtcggagcctccaaatgatcccgagaaagaagcatacgtcgttgacgttgtcctcaagtacctccgtgctgcaaagaaccccgtcatccttgtcgatgcttgtgctatccgtcatcgtgttcttgatgaggttcatgatctcatcgaaaagacaaacctccctgtctttgtcactcctatgggcaaaggtgctgttaacgaagaacacccgacatatggtggtgtctatgccggtgacggctcacatccgcctcaagttaaggacatggttgagtcttctgatttgatattgacaatcggtgctctcaagagcgacttcaacactgctggcttctcttaccgtacctcacagctgaacacgattgatctacacagcgaccactgcattgtcaaatactcgacatatccaggtgtccagatgaggggtgtgctgcgacaagtgattaagcagctcgatgcatctgagatcaacgctcagccagcgccagtcgtcgagaatgaagttgccaaaaaccgagataactcacccgtcattacacaagctttcttctggccgcgcgtgggagagttcctgaagaagaacgacatcgtcattaccgagactggaacagccaactttggcatctgggatactaagtttccctctggcgttactgcgctttctcaggtcctttggggaagcattggttggtccgttggtgcctgccaaggagccgttcttgcagccgccgatgacaacagcgatcgcagaactatcctctttgttggtgatggctcattccagctcactgctcaagaattgagcacaatgattcgtctcaagctgaagcccatcatctttgtcatctgcaacgatggctttaccattgaacgattcattcacggcatggaagccgagtacaacgacatcgcaaattgggacttcaaggctctggttgacgtctttggcggctctaagacggccaagaagttcgccgtcaagaccaaggacgagctggacagccttctcacagaccctacctttaacgccgcagaatgcctccagtttgtcgagctatatatgcccaaagaagatgctcctcgagcattgatcatgactgcagaagctagcgcgaggaacaatgccaagacagag

SEQ ID NO: 11 - protein, 458 aa, Unknown
  Fungal isolate from soil sample
RNGMLLHHTLGNGDFNIFANMSAQISCEVAKLTNPAEIATQIDHALRVCFIRSRPVYIMLPTDMVQAKVEGARLKEPIDLSEPPNDPEKEAYVVDVVLKYLRAAKNPVILVDACAIRHRVLDEVHDLIEKTNLPVFVTPMGKGAVNEEHPTYGGVYAGDGSHPPQVKDMVESSDLILTIGALKSDFNTAGFSYRTSQLNTIDLHSDHCIVKYSTYPGVQMRGVLRQVIKQLDASEINAQPAPVVENEVAKNRDNSPVITQAFFWPRVGEFLKKNDIVITETGTANFGIWDTKFPSGVTALSQVLWGSIGWSVGACQGAVLAAADDNSDRRTILFVGDGSFQLTAQELSTMIRLKLKPIIFVICNDGFTIERFIHGMEAEYNDIANWDFKALVDVFGGSKTAKKFAVKTKDELDSLLTDPTFNAAECLQFVELYMPKEDAPRALIMTAEASARNNAKTE

SEQ ID NO: 12 - DNA, 30 bp, Unknown
  CDS (1)...(30): Oligonucleotide for PCR amplicfication of GDC-1
tcccagatgccaaagttggctgttccagtc

SEQ ID NO: 13 - DNA, 2606 bp, Unknown
  CDS (168)...(2258): Fungal isolate from soil sample
caattgacgaggagtcgttgttttcctctttttctctctctcccgcatcgcgcgcgtggattgggccctttttatctttttctgcgatatcctcgactgagaacgacgacgacgagcacgacgacgacgacacaggcgacgactgcgaggcagcccccacagccgccatgatgctccgaagtcgccaggcctccaaggccctgagggccttgggccaggcacggcacttcacctcgacgacacagcccgccgccgtgcaggccccgagaaaggtcgcctccggacagcggaatcaagctaccgccgcgacggccacctctgccgcacccaatgtccgcgccacgccgagtcctgccttcaatgcggaggagcagcagcagcaaaaacacagccatgtccagccgctggtcaatccccagaagagcgacatggatgagtcgttcatcggcaagacgggcggcgaaatctttcacgaaatgatgctgagacaaggcgtcaagcacatctttggataccccggcggcgccatcttgcccgtcttcgatgccatctacaactcaaaacacttcgacttcatcctgcccagacacgagcagggcgccggccacatggccgagggctacgcccgcgcgtccggcaagcccggcgtcgtcctcgtcacctcgggccccggcgccaccaacgtcgtgaccccaatgcaggacgccctgtccgacggcacgccactcgtcgtcttttgcggccaggtcccgacctcggccatcggcagcgatgccttccaggaggccgacgtcgtcggcatctcccgcgcctgcaccaagtggaacgtcatggtcaagaacgtcgcggagctgccgcggagaatcaacgaggcctttgagattgccacgagcggtcgccccggccccgtcctcgtcgacctgcccaaggacgtcaccgccggcatcctgaggagagccatccccacggagacggccctgcccgcgctgccgagcgccgcctcgcgcgccgccatggagtcgagccggaaacacctcgagcacaccatcaagcgcgtcgccgacctcgtcaacaaggccaagcagccagtcatctacgccggccagggcatcatccagtccgagggcgggcccgagctcctcaaggagctggccgacaaggcctccatccccgtcaccacgaccctccagggcctcggcggcttcgacgagctcgacgagaagtcgctgcacatgctcggcatgcacggctcggcctacgccaacatggccatgcaggaggccgacctcatcatcgccctcggcgcgcgcttcgacgaccgcgtcaccctcaacgtggccaagttcgcgcctggcgcgagggccgccgcggccgagaagcgcggcggcatcgtccacttcgaggtgatgcccaagaacatcaacaaggtggtgcaggccaccgaggccgtcgagggcaacgtcggcagcaacctcaagctcctgctgcccgaggtgcaggccaagacgatggacgaccgcaaggagtggttcggcaagatcaacgagtggaagaagaagtggccgctgtcgcactacgagcgtgcggagcgccacgggctcatcaagccgcagaccctcatcgaggagctgagcaagctgacggcggaccgcaaggacaagacgtacattgccaccggcgtcggacagcaccagatgtggacggcccagcacttccggtggaggcacccgcgcagcatgatcacgtcgggtggtctcggcactatgggcttcggtctgccggctgccatcggtgccaaggtcgcgcagccggacgccctcgtcttcgatatcgatggcgacgcgtcatttggcatgaccctgacggagctggccacggcggcgcagttcaacattggcgtcaaggtcattgtcctcaacaacgaggagcagggcatggtaacgcagtggcagaacctcttctacgaggaccgctacgcgcacacgcaccaggtcaaccctgatttcatgaagctggccgagtcgatgcgcgtccagggccggcgatgcgtggaccccgaggacgtggtcgacagcctgaagtggctgatcaacactgacggcccggccctgctggaggttgtcacggacaagaaggtgcccgtcctgcccatggtgccggcgggctcggccctgcacgagtttttggtgtttgacggagaaaaggacaagaagcgacgagagctgatgcgggaaaggacctcgggcctgcacggctagccgcagcacacggggcggattagcagcacccgacgacgggcatccatccatcaatcatcttctagtcatgttcttttcatacctcttactggcggagttttgtgcagttaangcaaatccgggcgcgaagcacaaaaagttggaggaggagcagcgccgaacggcggcgcggtggtagcacaggggtggcaatgtgacggcgggtcgaagagcccgggcatggcagagtagggcggttggttcccatgaggcgagcgagccgcgcgcgggcttgcggacggacacaaacaaacaatgaatgaccatttttccgagacgtgaaaaaaaaaaaaaaaaaaaaaaaaaaaa

SEQ ID NO: 14 - DNA, 2088 bp, Unknown
  CDS (1)...(2088): Fungal isolate from soil sample
atgatgctccgaagtcgccaggcctccaaggccctgagggccttgggccaggcacggcacttcacctcgacgacacagcccgccgccgtgcaggccccgagaaaggtcgcctccggacagcggaatcaagctaccgccgcgacggccacctctgccgcacccaatgtccgcgccacgccgagtcctgccttcaatgcggaggagcagcagcagcaaaaacacagccatgtccagccgctggtcaatccccagaagagcgacatggatgagtcgttcatcggcaagacgggcggcgaaatctttcacgaaatgatgctgagacaaggcgtcaagcacatctttggataccccggcggcgccatcttgcccgtcttcgatgccatctacaactcaaaacacttcgacttcatcctgcccagacacgagcagggcgccggccacatggccgagggctacgcccgcgcgtccggcaagcccggcgtcgtcctcgtcacctcgggccccggcgccaccaacgtcgtgaccccaatgcaggacgccctgtccgacggcacgccactcgtcgtcttttgcggccaggtcccgacctcggccatcggcagcgatgccttccaggaggccgacgtcgtcggcatctcccgcgcctgcaccaagtggaacgtcatggtcaagaacgtcgcggagctgccgcggagaatcaacgaggcctttgagattgccacgagcggtcgccccggccccgtcctcgtcgacctgcccaaggacgtcaccgccggcatcctgaggagagccatccccacggagacggccctgcccgcgctgccgagcgccgcctcgcgcgccgccatggagtcgagccggaaacacctcgagcacaccatcaagcgcgtcgccgacctcgtcaacaaggccaagcagccagtcatctacgccggccagggcatcatccagtccgagggcgggcccgagctcctcaaggagctggccgacaaggcctccatccccgtcaccacgaccctccagggcctcggcggcttcgacgagctcgacgagaagtcgctgcacatgctcggcatgcacggctcggcctacgccaacatggccatgcaggaggccgacctcatcatcgccctcggcgcgcgcttcgacgaccgcgtcaccctcaacgtggccaagttcgcgcctggcgcgagggccgccgcggccgagaagcgcggcggcatcgtccacttcgaggtgatgcccaagaacatcaacaaggtggtgcaggccaccgaggccgtcgagggcaacgtcggcagcaacctcaagctcctgctgcccgaggtgcaggccaagacgatggacgaccgcaaggagtggttcggcaagatcaacgagtggaagaagaagtggccgctgtcgcactacgagcgtgcggagcgccacgggctcatcaagccgcagaccctcatcgaggagctgagcaagctgacggcggaccgcaaggacaagacgtacattgccaccggcgtcggacagcaccagatgtggacggcccagcacttccggtggaggcacccgcgcagcatgatcacgtcgggtggtctcggcactatgggcttcggtctgccggctgccatcggtgccaaggtcgcgcagccggacgccctcgtcttcgatatcgatggcgacgcgtcatttggcatgaccctgacggagctggccacggcggcgcagttcaacattggcgtcaaggtcattgtcctcaacaacgaggagcagggcatggtaacgcagtggcagaacctcttctacgaggaccgctacgcgcacacgcaccaggtcaaccctgatttcatgaagctggccgagtcgatgcgcgtccagggccggcgatgcgtggaccccgaggacgtggtcgacagcctgaagtggctgatcaacactgacggcccggccctgctggaggttgtcacggacaagaaggtgcccgtcctgcccatggtgccggcgggctcggccctgcacgagtttttggtgtttgacggagaaaaggacaagaagcgacgagagctgatgcgggaaaggacctcgggcctgcacggc

SEQ ID NO: 15 - protein, 696 aa, Unknown
  Fungal isolate from soil sample
MMLRSRQASKALRALGQARHFTSTTQPAAVQAPRKVASGQRNQATAATATSAAPNVRATPSPAFNAEEQQQQKHSHVQPLVNPQKSDMDESFIGKTGGEIFHEMMLRQGVKHIFGYPGGAILPVFDAIYNSKHFDFILPRHEQGAGHMAEGYARASGKPGVVLVTSGPGATNVVTPMQDALSDGTPLVVFCGQVPTSAIGSDAFQEADVVGISRACTKWNVMVKNVAELPRRINEAFEIATSGRPGPVLVDLPKDVTAGILRRAIPTETALPALPSAASRAAMESSRKHLEHTIKRVADLVNKAKQPVIYAGQGIIQSEGGPELLKELADKASIPVTTTLQGLGGFDELDEKSLHMLGMHGSAYANMAMQEADLIIALGARFDDRVTLNVAKFAPGARAAAAEKRGGIVHFEVMPKNINKVVQATEAVEGNVGSNLKLLLPEVQAKTMDDRKEWFGKINEWKKKWPLSHYERAERHGLIKPQTLIEELSKLTADRKDKTYIATGVGQHQMWTAQHFRWRHPRSMITSGGLGTMGFGLPAAIGAKVAQPDALVFDIDGDASFGMTLTELATAAQFNIGVKVIVLNNEEQGMVTQWQNLFYEDRYAHTHQVNPDFMKLAESMRVQGRRCVDPEDVVDSLKWLINTDGPALLEVVTDKKVPVLPMVPAGSALHEFLVFDGEKDKKRRELMRERTSGLHG

SEQ ID NO: 16 - protein, 563 aa, Saccharomyces cerevisiae
MSEITLGKYLFERLKQVNVNTVFGLPGDFNLSLLDKIYEVEGMRWAGNANELNARYAADGYARIKGMSCIITTFGVGELSALNGIAGSYAEHVGVLHVVGVPSISSQAKQLLLHHTLGNGDFTVFHRMSANISETTAMITDICTAPAEIDRCIRTTYVTQRPVYLGLPANLVDLNVPAKLLQTPIDMSLKPNDAESEKEVIDTILVLAKDAKNPVILADACCSRHDVKAETKKLIDLTQFPAFVTPMGKGSISEQHPRYGGVYVGTLSKPEVKEAVESADLILSVGALLSDFNTGSFSYSYKTKNIVEFHSDHMKIRNATFPGVQMKFVLQKLLTNIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEVMLPVFDAPQNLVEQAKLTAATNAKQ

SEQ ID NO: 17 - protein, 550 aa, Salmonella typhimurium
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVLQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 18 - protein, 568 aa, Zymomonas mobilis
MSYTVGTYLAERLVQIGLKHHFAVAGDYNLVLLDNLLLNKNMEQVYCCNELNCGFSAEGYARAKGAAAAVVTYSVGAHSAFDAIGGAYAENLPVILISGAPNNNDHAAGHVLHHALGKTDYHYQLEMAKNITAAAEAIYTPEEAPAKIDHVIKTALAKKKPVYLEIACNIASMPCAAPGPASALFNDEASDEASLNAAVDETLKFIANRDKVAVLVGSKLRAAGAEEAAVKFTDALGGAVATMAAAKSFFPEENPHYIGTSWGEVSYPGVEKTMKEADAVIALAPVFNDYSTTGWTDIPDPKKLVLAEPRSVVVRRIRFPSVHLKDYLTRLAQKVSKKTGSLDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAAKGLKAKTGGELAEAIKVALANTDGPTLIECFIGREDCTEELVKWGKRVAAANSRKPVNKLL

SEQ ID NO: 19 - protein, 687 aa, Saccharomyces cerevisiae [sequence omitted]

SEQ ID NO: 20 - protein, 686 aa, Magnaporthe grisea
MLRTVGRKALRGSSKGCSRTISTLKPATATIAKPGSRTLSTPATATATAPRTKPSASFNARRDPQPLVNPRSGEADESFIGKTGGEIFHEMMLRQNVKHIFGYPGGAILPVFDAIYNSKHIDFVLPKHEQGAGHMAEGYARASGKPGVVLVTSGPGATNVITPMADALADGTPLVVFSGQVVTSDIGSDAFQEADVIGISRSCTKWNVMVKSADELPRRINEAFEIATSGRPGPVLVDPAKDVTASVLRRAIPTETSIPSISAAARAVQEAGRKQLEHSIKRVADLVNIAKKPVIYAGQGVILSEGGVELLKALADKASIPVTTTLHGLGAFDELDEKALHMLGMHGSAYANMSMQEADLIIALGGRFDDRVTGSIPKFAPAAKLAAAEGRGGIVHFEIMPKNINKVVQATEAIEGDVASNLKLLLPKIEQRSMTDRKEWFDQIKEWKEKWPLSHYERAERSGLIKPQTLIEELSNLTADRKDMTYITTGVGQHQMWTAQHFRWRHPRSMITSGGLGTMGYGLPAAIGAKVARPDALVIDIDGDASFNMTLTELSTAAQFNIGVKVIVLNNEEQGMVTQWQNLFYEDRYSHTHQRNPDFMKLADAMDVQHRRVSKPDDVGDALTWLINTDGPALLEVMTDKKVPVLPMVPGGNGLHEFITFDASKDKQRRELMRARTNGLHGRTAV

SEQ ID NO: 21 - DNA, 1728 bp, Unknown
  Fungal isolate from soil sample
atggccagcatcaacatcagggtgcagaatctcgagcaacccatggacgttgccgagtatcttttccggcgtctccacgaaatcggcattcgctccatccacggtcttccaggcgattacaaccctcttgccctcgactatttgccatcatgtggcctgagatgggttggcagcgtcaacgaactcaatgctgcttatgctgctgatggctatgcccgcgtcaagcagatgggagctctcatcaccacttttggagtgggagagctctcagccatcaatggcgttgccggtgccttttcggaacacgtcccagtcgttcacattgttggctgcccttccactgcctcgcagcgaaacggcatgctcctccaccacacgcttggaaacggcgacttcaacatctttgccaacatgagcgctcaaatctcttgcgaagtggccaagctcaccaaccctgccgaaattgcgacccagatcgaccatgccctccgcgtttgcttcattcgttctcggcccgtctacatcatgcttcccaccgatatggtccaggccaaagtagaaggtgccagactcaaggaaccaattgacttgtcggagcctccaaatgatcccgagaaagaagcatacgtcgttgacgttgtcctcaagtacctccgtgctgcaaagaaccccgtcatccttgtcgatgcttgtgctatccgtcatcgtgttcttgatgaggttcatgatctcatcgaaaagacaaacctccccgtctttgtcactcctatgggcaaaggtgctgttaacgaagaacacccgacatatggtggtgtctatgccggtgacggctcacatccgcctcaagttaaggacatggttgagtcttctgatttgatattgacaatcggtgctctcaagagcgacttcaacactgctggcttctcttaccgtacctcacagctgaacacgattgatctacacagcgaccactgcattgtcaaatactcgacatatccaggtgtccagatgaggggtgtgctgcgacaagtgattaagcagctcgatgcatctgagatcaacgctcagccagcgccagtcgtcgagaatgaagttgccaaaaaccgagataactcacccgtcattacacaagctttcttctggccgcgcgtgggagagttcctgaagaagaacgacatcgtcattaccgagactggaacagccaactttggcatctgggatactaagtttccctctggcgttactgcgctttctcaggtcctttggggaagcattggttggtccgttggtgcctgccaaggagccgttcttgcagccgccgatgacaacagcgatcgcagaactatcctctttgttggtgatggctcattccagctcactgctcaagaattgagcacaatgattcgtctcaagctgaagcccatcatctttgtcatctgcaacgatggctttaccattgaacgattcattcacggcatggaagccgagtacaacgacatcgcaaattgggacttcaaggctctggttgacgtctttggcggctctaagacggccaagaagttcgccgtcaagaccaaggacgagctggacagccttctcacagaccctacctttaacgccgcagaatgcctccagtttgtcgagctatatatgcccaaagaagatgctcctcgagcattgatcatgacggcagaagctagcgcgaggaacaatgccaagacagagtaa

SEQ ID NO: 22 - protein, 575 aa, Unknown
  Fungal isolate from soil sample
MASINIRVQNLEQPMDVAEYLFRRLHEIGIRSIHGLPGDYNPLALDYLPSCGLRWVGSVNELNAAYAADGYARVKQMGALITTFGVGELSAINGVAGAFSEHVPVVHIVGCPSTASQRNGMLLHHTLGNGDFNIFANMSAQISCEVAKLTNPAEIATQIDHALRVCFIRSRPVYIMLPTDMVQAKVEGARLKEPIDLSEPPNDPEKEAYVVDVVLKYLRAAKNPVILVDACAIRHRVLDEVHDLIEKTNLPVFVTPMGKGAVNEEHPTYGGVYAGDGSHPPQVKDMVESSDLILTIGALKSDFNTAGFSYRTSQLNTIDLHSDHCIVKYSTYPGVQMRGVLRQVIKQLDASEINAQPAPVVENEVAKNRDNSPVITQAFFWPRVGEFLKKNDIVITETGTANFGIWDTKFPSGVTALSQVLWGSIGWSVGACQGAVLAAADDNSDRRTILFVGDGSFQLTAQELSTMIRLKLKPIIFVICNDGFTIERFIHGMEAEYNDIANWDFKALVDVFGGSKTAKKFAVKTKDELDSLLTDPTFNAAECLQFVELYMPKEDAPRALIMTAEASARNNAKTE

SEQ ID NO: 23 - DNA, 1728 bp, Unknown
  Fungal isolate from soil sample
atggccagcatcaacatcagggtgcagaatctcgagcaacccatggacgttgccgagtatcttttccggcgtctccacgaaatcggcattcgctccatccacggtcttccaggcgattacaaccttcttgccctcgactatttgccatcatgtggcctgagatgggttggcagcgtcaacgaactcaatgctgcttatgctgctgatggctatgcccgcgtcaagcagatgggagctctcatcaccacttttggagtgggagagctctcagccatcaatggcgttgccggtgccttttcggaacacgtcccagtcgttcacattgttggctgcccttccactgcctcgcagcgaaacggcatgctcctccaccacacgcttggaaacggcgacttcaacatctttgccaacatgagcgctcaaatctcttgcgaagtggccaagctcaccaaccctgccgaaattgcgacccagatcgaccatgccctccgcgtttgcttcattcgttctcggcccgtctacatcatgcttcccaccgatatggtccaggccaaagtagaaggtgccagactcaaggaaccaattgacttgtcggagcctccaaatgatcccgagaaagaagcatacgtcgttgacgttgtcctcaagtacctccgtgctgcaaagaaccccgtcatccttgtcgatgcttgtgctatccgtcatcgtgttcttgatgaggttcatgatctcatcgaaaagacaaacctccccgtctttgtcactcctatgggcaaaggtgctgttaacgaagaacacccgacatatggtggtgtctatgccggtgacggctcacatccgcctcaagttaaggacatggttgagtcttctgatttgatattgacaatcggtgctctcaagagcgacttcaacactgctggcttctcttaccgtacctcacagctgaacacgattgatctacacagcgaccactgcattgtcaaatactcgacatatccaggtgtccagatgaggggtgtgctgcgacaagtgattaagcagctcgatgcatctgagatcaacgctcagccagcgccagtcgtcgagaatgaagttgccaaaaaccgagataactcacccgtcattacacaagctttcttctggccgcgcgtgggagagttcctgaagaagaacgacatcgtcattaccgagactggaacagccaactttggcatctgggatactaagtttccctctggcgttactgcgctttctcaggtcctttggggaagcattggttggtccgttggtgcctgccaaggagccgttcttgcagccgccgatgacaacagcgatcgcagaactatcctctttgttggtgatggctcattccagctcactgctcaagaattgagcacaatgattcgtctcaagctgaagcccatcatctttgtcatctgcaacgatggctttaccattgaacgattcattcacggcatggaagccgagtacaacgacatcgcaaattgggacttcaaggctctggttgacgtctttggcggctctaagacggccaagaagttcgccgtcaagaccaaggacgagctggacagccttctcacagaccctacctttaacgccgcagaatgcctccagtttgtcgagctatatatgcccaaagaagatgctcctcgagcattgatcatgacggcagaagctagcgcgaggaacaatgccaagacagagtaa

SEQ ID NO: 24 - protein, 575 aa, Unknown
  Fungal isolate from soil sample
MASINIRVQNLEQPMDVAEYLFRRLHEIGIRSIHGLPGDYNLLALDYLPSCGLRWVGSVNELNAAYAADGYARVKQMGALITTFGVGELSAINGVAGAFSEHVPVVHIVGCPSTASQRNGMLLHHTLGNGDFNIFANMSAQISCEVAKLTNPAEIATQIDHALRVCFIRSRPVYIMLPTDMVQAKVEGARLKEPIDLSEPPNDPEKEAYVVDVVLKYLRAAKNPVILVDACAIRHRVLDEVHDLIEKTNLPVFVTPMGKGAVNEEHPTYGGVYAGDGSHPPQVKDMVESSDLILTIGALKSDFNTAGFSYRTSQLNTIDLHSDHCIVKYSTYPGVQMRGVLRQVIKQLDASEINAQPAPVVENEVAKNRDNSPVITQAFFWPRVGEFLKKNDIVITETGTANFGIWDTKFPSGVTALSQVLWGSIGWSVGACQGAVLAAADDNSDRRTILFVGDGSFQLTAQELSTMIRLKLKPIIFVICNDGFTIERFIHGMEAEYNDIANWDFKALVDVFGGSKTAKKFAVKTKDELDSLLTDPTFNAAECLQFVELYMPKEDAPRALIMTAEASARNNAKTE

(19) United States
(12) Patent Application Publication
(10) Pub. No.: US 2005/0204436 A1
(43) Pub. Date: Sep. 15, 2005

(54) Methods to confer herbicide resistance

(75) Inventors: Philip E. Hammer, Cary, NC (US); Todd K. Hinson, Rougemont, NC (US); Nicholas B. Duck, Apex, NC (US); Michael G. Koziel, Raleigh, NC (US)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Athenix Corporation, Durham, NC (US)

(21) Appl. No.: 10/797,270

(22) Filed: Mar. 10, 2004

(51) Int. Cl. C12N 15/82; A01H 5/10; C12N 15/79; A01H 1/00

(52) U.S. Cl. 800/300; 800/278; 800/298; 435/252.3

(57) ABSTRACT

Compositions and methods for conferring herbicide resistance to plant cells and bacterial cells are provided. The methods comprise transforming the cells with nucleotide sequences encoding herbicide resistance genes. In particular, herbicide resistance is conferred by expression of proteins with homology to decarboxylase enzymes. Compositions comprise transformed plants, plant tissues, and seeds, as well as transformed bacterial cells.

### CROSS-REFERENCE TO RELATED APPLICATIONS

The present application claims the benefit of U.S. Provisional Application Ser. No. 60/453,148, filed Mar. 10, 2003, the contents of which are herein incorporated by reference in their entirety.

### FIELD OF THE INVENTION

Methods to confer herbicide resistance to cells, particularly glyphosate resistance, are provided. These methods are especially useful with plant and bacterial cells.

### BACKGROUND OF THE INVENTION

N-phosphonomethylglycine, commonly referred to as glyphosate, is an important agronomic chemical. Glyphosate inhibits the enzyme that converts phosphoenolpyruvic acid (PEP) and 3-phosphoshikimic acid to 5-enolpyruvyl-3-phosphoshikimic acid. Inhibition of this enzyme (5-enolpyruvylshikimate-3-phosphate synthase; referred to herein as “EPSP synthase”) kills plant cells by shutting down the shikimate pathway, thereby inhibiting aromatic acid biosynthesis.

Since glyphosate-class herbicides inhibit aromatic amino acid biosynthesis, they not only kill plant cells, but are also toxic to bacterial cells. Glyphosate inhibits many bacterial EPSP synthases, and thus is toxic to these bacteria. However, certain bacterial EPSP synthases may have a high tolerance to glyphosate.

Plant cells resistant to glyphosate toxicity can be produced by transforming plant cells to express glyphosate-resistant EPSP synthases. A mutated EPSP synthase from Salmonella typhimurium strain CT7 confers glyphosate resistance in bacterial cells, and confers glyphosate resistance on plant cells (U.S. Pat. Nos. 4,535,060, 4,769,061, and 5,094,945). Thus, there is a precedent for the use of glyphosate-resistant bacterial EPSP synthases to confer glyphosate resistance upon plant cells.

An alternative method to generate target genes resistant to a toxin (such as an herbicide) is to identify and develop enzymes that result in detoxification of the toxin to an inactive or less active form. This can be accomplished by identifying enzymes that encode resistance to the toxin in a toxin-sensitive test organism, such as a bacterium.

Castle et al. (WO 02/36782 A2) describe proteins (glyphosate N-acetyltransferases) that are described as modifying glyphosate by acetylation of a secondary amine to yield N-acetylglyphosate.

Barry et al. (U.S. Pat. No. 5,463,175) describes genes encoding an oxidoreductase (GOX), and states that GOX proteins degrade glyphosate by removing the phosphonate residue to yield amino methyl phosphonic acid (AMPA). This suggests that glyphosate resistance can also be conferred, at least partially, by removal of the phosphonate group from glyphosate. However, the resulting compound (AMPA) appears to provide reduced but measurable toxicity upon plant cells. Barry describes the effect of AMPA accumulation on plant cells as resulting in effects including chlorosis of leaves, infertility, stunted growth, and death. Barry (U.S. Pat. No. 6,448,476) describes plant cells expressing an AMPA-N-acetyltransferase (phnO) to detoxify AMPA.

Phosphonates, such as glyphosate, can also be degraded by cleavage of C—P bond by a C—P lyase. Wacket et al. (1987) J. Bacteriol. 169:710-717) described strains that utilize glyphosate as a sole phosphate source. Kishore et al. (1987) J. Biol. Chem. 262:12164-12168 and Shinabarger et al. (1986) J. Bacteriol. 168:702-707 describe degradation of glyphosate by C—P Lyase to yield glycine and inorganic phosphate.

While several strategies are available for detoxification of toxins, such as the herbicide glyphosate, as described above, new activities capable of degrading glyphosate are useful. Novel genes and genes conferring glyphosate resistance by novel mechanisms of action would be of additional usefulness. Single genes conferring glyphosate resistance by formation of non-toxic products would be especially useful.

Further, genes conferring resistance to other herbicides, such as the sulfonylureas or imidazolinones, are useful. The sulfonylurea and imidazolinine herbicides are widely used in agriculture because of their efficacy at low use rates against a broad spectrum of weeds, lack of toxicity to mammals, and favorable environmental profile (Saari et al. (1994) p. 83-139 in: Herbicide Resistance in Plants: Biology and Biochemistry. S. Powles and J. Holtum eds. Lewis Publishers, Inc., Boca Raton, Fla.). These herbicides act by inhibiting acetohydroxyacid synthase (AHAS, also known as acetolactate synthase) and thereby preventing the biosynthesis of the branched-chain amino acids valine, leucine and isoleucine.

Current methods of herbicide tolerance confer upon a plant tolerance to herbicides with a particular target or mode of action. However, repeated and extensive use of herbicides with a single mode of action can result in the selection of tolerant weed species (Saari et al., supra). Crop plants which are resistant to more than one class of herbicides (with different modes of action) provide growers with flexibility in weed control options and are useful in preventing/managing the emergence of resistant weed populations. Plants containing a single trait that conferred tolerance to more than one class of herbicide would be particularly desirable. Thus, genes encoding resistance to more than one class of herbicide are useful.

Thus, methods that result in degradation of herbicides to non-toxic forms are desired. Further, methods that achieve sufficient degradation to allow cells to grow in otherwise toxic concentrations of herbicide (“herbicide resistance”) are desired. Methods that confer “herbicide resistance” through the expression of a single protein would be preferred, since expression of a single protein in a cell such as a plant cell is technically less complex than the expression of multiple proteins. Further, in some instances, methods for conferring herbicide resistance that are compatible with, and/or improve the efficacy of other methods of conferring herbicide resistance, are desirable.

### SUMMARY OF INVENTION

Compositions and methods for conferring herbicide resistance to bacteria, plants, plant cells, tissues and seeds are provided. In particular, herbicide resistance is conferred by expression of proteins with homology to decarboxylase enzymes. In one embodiment, the herbicide is a glyphosate herbicide. In addition, the expressed protein may result in increased tolerance of the cell to more than one herbicide. Compositions comprise transformed bacteria, plants, plant cells, tissues, and seeds.

Decarboxylase enzymes that could be useful in conferring herbicide resistance include, but are not limited to, a pyruvate decarboxylase, a benzoylformate decarboxylase, an oxalyl-CoA decarboxylase, a 2-oxoglutarate decarboxylase, an indolepyruvate decarboxylase, a 5-guanidino-2-oxopentanoate decarboxylase, a phenylglyoxylate dehydrogenase (acylating), a pyruvate dehydrogenase (cytochrome), a pyruvate oxidase, a pyruvate dehydrogenase (lipoamide), an oxoglutarate dehydrogenase (lipoamide), a transketolase, a formaldehyde transketolase, an acetoin-ribose-5-phosphate transaldolase, a tartronate-semialdehyde synthase, a phosphoketolase, a fructose-6-phosphate phosphoketolase, a benzoin aldolase, a 2-hydroxy-3-oxoadipate synthase, an acetolactate synthase, an 1-deoxy-C-xylulose 5-phosphate synthase, and a sulfoacetaldehyde lyase.

### DESCRIPTION OF FIGURES

FIGS. 1A and B show an alignment of GDC-1 (SEQ ID NO:22) and GDC-2 (SEQ ID NO:15) to pyruvate decarboxylase of Saccharomyces cerevesiae (SEQ ID NO:16), a putative indole-3-pyruvate decarboxylase from Salmonella typhimurium (SEQ ID NO:17), pyruvate decarboxylase (EC 4.1.1.1) from Zymomonas mobilis (SEQ ID NO:18), acetolactate synthase from Saccharomyces cerevesiae (SEQ ID NO:19), and acetolactate synthase from Magnaporthe grisea (SEQ ID NO:20). The alignment shows the most highly conserved amino acid residues highlighted in black, and highly conserved amino acid residues highlighted in gray.

FIG. 2A shows growth of GDC-1 expressing cells at various concentrations of glyphosate as compared to vector and media only controls at 42 hours. FIG. 2B shows growth of GDC-2 expressing cells at various concentrations of glyphosate as compared to vector and media only controls at 42 hours. Growth was measured by absorbance at 600 nm.

FIG. 3A shows the HPLC column elution profile of C¹⁴ from a sample not incubated with GDC-1. FIG. 3B shows the HPLC column elution profile of C¹⁴ after incubation with 100 ng GDC-1.

### DETAILED DESCRIPTION

The present invention is drawn to compositions and methods for conferring resistance to an herbicide in a cell, particularly in a plant cell or a bacterial cell. The methods involve transforming the cell with a nucleotide sequence encoding an herbicide resistance gene. In particular, the methods of the invention are useful for preparing plant and bacterial cells that show increased tolerance to the herbicide glyphosate. Thus, compositions include transformed plants, plant cells, plant tissues and seeds as well as transformed bacterial cells.

Definitions

“Glyphosate” includes any herbicidal form of N-phosphonomethylglycine (including any salt thereof) and other forms that result in the production of the glyphosate anion in planta.

“Glyphosate (or herbicide) resistance-conferring decarboxylase” or “GDC” includes a DNA segment that encodes all or part of a glyphosate (or herbicide) resistance protein. This includes DNA segments that are capable of expressing a protein that confers glyphosate (herbicide) resistance to a cell.

An “herbicide resistance protein” or an “herbicide resistance protein molecule” or a protein resulting from expression of an “herbicide resistance-encoding nucleic acid molecule” includes proteins that confer upon a cell the ability to tolerate a higher concentration of an herbicide than cells that do not express the protein, or to tolerate a certain concentration of an herbicide for a longer time than cells that do not express the protein.

A “glyphosate resistance protein”, includes a protein that confers upon a cell the ability to tolerate a higher concentration of glyphosate than cells that do not express the protein, or to tolerate a certain concentration of glyphosate for a longer time than cells that do not express the protein. By “tolerate” or “tolerance” is intended either to survive, or to carry out essential cellular functions such as protein synthesis and respiration in a manner that is not readily discernable from untreated cells.

By “decarboxylase” is intended a protein, or gene encoding a protein, whose catalytic mechanism can include cleavage and release of a carboxylic acid. This includes enzymes that liberate CO₂, such as pyruvate decarboxlyases, acetolactate synthases, and orthinine decarboxylases, as well as enzymes that liberate larger carboxylic acids, as illustrated in Table 1. “Decarboxylase” includes proteins that utilize thiamine pyrophoshate as a cofactor in enzymatic catalysis. Many such decarbolyases also utilize other cofactors, such as FAD.

By “TPP-binding domain” is intended a region of conserved amino acids present in enzymes that are capable of utilizing TPP as a cofactor.

“Plant tissue” includes all known forms of plants, including undifferentiated tissue (e.g. callus), suspension culture cells, protoplasts, plant cells including leaf cells, root cells, and phloem cells, plant seeds, pollen, propagules, embryos and the like.

“Plant expression cassette” includes DNA constructs that are capable of resulting in the expression of a protein from an open reading frame in a plant cell. Typically these contain a promoter and a coding sequence. Often, such constructs will also contain a 3′ untranslated region. Such constructs may contain a ‘signal sequence’ or ‘leader sequence’ to facilitate co-translational or post-translational transport of the peptide to certain intracellular structures such as the chloroplast (or other plastid), endoplasmic reticulum, or Golgi apparatus.

“Signal sequence” includes sequences that are known or suspected to result in co-translational or post-translational peptide transport across the cell membrane. In eukaryotes, this typically involves secretion into the Golgi apparatus, with some resulting glycosylation.

“Leader sequence” includes any sequence that when translated, results in an amino acid sequence sufficient to trigger co-translational transport of the peptide chain to a sub-cellular organelle. Thus, this includes leader sequences targeting transport and/or glycosylation by passage into the endoplasmic reticulum, passage to vacuoles, plastids including chloroplasts, mitochondria, and the like.

“Plant transformation vector” includes DNA molecules that are necessary for efficient transformation of a plant cell. Such a molecule may consist of one or more plant expression cassettes, and may be organized into more than one ‘vector’ DNA molecules. For example, binary vectors are plant transformation vectors that utilize two non-contiguous DNA vectors to encode all requisite cis- and trans-acting fuictions for transformation of plant cells (Hellens and Mullineaux (2000) Trends in Plant Science 5:446-451).

“Vector” refers to a nucleic acid construct designed for transfer between different host cells. “Expression vector” refers to a vector that has ability to incorporate, integrate and express heterologous DNA sequences or fragments in a foreign cell.

“Transgenic plants” or “transformed plants” or “stably transformed plants or cells or tissues” refers to plants that have incorporated or integrated exogenous or endogenous nucleic acid sequences or DNA fragments or chimeric nucleic acid sequences or fragments.

“Heterologous” generally refers to the nucleic acid sequences that are not endogenous to the cell or part of the native genome in which they are present, and have been added to the cell by infection, transfection, microinjection, electroporation, microprojection, or the like.

“Promoter” refers to a nucleic acid sequence that functions to direct transcription of a downstream coding sequence. The promoter together with other transcriptional and translational regulatory nucleic acid sequences (also termed as “control sequences”) are necessary for the expression of a DNA sequence of interest.

Various aspects of the invention are described in further detail in the following subsections.

Decarboxylases

Decarboxylation is a general class of chemical reactions, generally defined as a reaction that results in cleavage of a carbon-carbon bond, resulting in the liberation of a new carbon, often in the form of carbon dioxide (CO₂). A thorough description of the biochemical mechanism of decarboxylation is provided in the following references, herein incorporated by reference (Jorday (1999) FEBS letters 457:298-301; Pohl (1997) Adv. Biochem. Eng. Biotechnol 58:15-43).

Decarboxylases are also capable of performing condensation reactions (reactions that combine two compounds). Typically such reactions are known in the art as carboligation reactions, and typically result in production of hydroxy ketones. Decarboxylases in general, including pyruvate decarboxylases and acetolactate synthases, are known to be able to perform carboligation reactions on a wide variety of substrates (for review, see Ward and Singh (2000) Current Opinions in Biotechnology 11:520-526, and Ohta and Sugai (2000) “Enzyme-mediated Decarboxylation Reactions in organic synthesis” in Stereoselective Biocatalysis, Patel, R. N., ed, Marcel Deckker, Inc., references therein).

Many decarboxylation enzymes utilize the cofactor thiamine pyrophosphate (referred to herein as “TPP”). TPP facilitates many enzyme reactions, typically those involving transfer of aldehyde groups from a donor molecule to an acceptor molecule. A well-known example of a decarboxylation reaction involving TPP as a cofactor is the conversion of pyruvate to acetaldehyde and CO₂ by the enzyme pyruvate decarboxylase. Acetolactate synthases are another example of a class of decarboxylating enzymes that utilize TPP as a cofactor. Examples of other reactions that utilize TPP as a cofactor include dehydrogenations, such as the reaction catalyzed by pyruvate dehydrogenase, and α-ketoglutarate dehydrogenase.

Thus, the coenzyme TPP is a valuable cofactor, important for catalytic processes. Analysis of amino acid sequences of known TPP-utilizing enzymes has allowed the identification of amino acid regions common to each class of TPP-utilizing proteins. Enzymes that are capable of utilizing TPP as a cofactor share several regions of amino acid conservation, referred to herein as “TPP-binding domains”. These regions are often referred to as the N-terminal domain, central domain, and C-terminal domain, in reference to their position within the amino acid sequence (see for example, Hawkins et al. (1989) FEBS Letters 255:77-82; Arjunan et al. (1996) J. Mol. Biol. 256:590-600; Barilan et al. (2001) Biochemistry 40:11946-11954). Thus, pyruvate decarboxylase, pyruvate dehydrogenase, α-ketoglutarate dehydrogenase, and acetolactate synthase each contain TPP-binding domains. Further, the amino acid conservation shared by TPP-binding proteins can be identified by comparison of the amino acid sequence of a new protein with the amino acid sequence of known TPP-binding proteins.

Aside from the presence of conserved domains, decarboxylase enzymes can also share significant amino acid homology in regions of their amino acid sequence other than the conserved domains. Thus, a high degree of amino acid conservation is suggestive of similar functional role.

Co-pending U.S. Application entitled “GDC-1 Genes Conferring Herbicide Resistance”, filed concurrently herewith, and incorporated herein by reference, describes the identification of a gene sequence referred to therein as GDC-1. The sequence of GDC-1 encodes an herbicide resistance protein, conferring resistance to the herbicide glyphosate. Co-pending U.S. Application entitled “GDC-2 Genes Conferring Herbicide Resistance”, filed concurrently herewith, and incorporated herein by reference, describes the identification of a gene sequence referred to therein as GDC-2. The sequence of GDC-2 encodes an herbicide resistance protein, conferring resistance to the herbicide glyphosate. GDC-1 and GDC-2 contain TPP-binding domains. While not being bound by any particular mechanism of action, the homology of the protein sequences of GDC-1 and GDC-2 herbicide tolerance-conferring genes to TPP-binding decarboxylases, as well as biochemical data provided herein, suggests that GDC-1 and/or GDC-2 encode herbicide tolerance by reactions involving the cofactor TPP.

Thus, by identifying genes encoding proteins with a high homology to known decarboxylases, one is likely to identify previously unknown decarboxylases. Many of these decarboxylases may be capable of functioning to detoxify herbicides such as glyphosate.

Having provided that proteins containing TPP-binding domains are capable of conferring resistance to glyphosate, it is understood that one skilled in the art could measure the decarboxylation activity of any of these proteins, for example by incubating a purified, semi-purified, or crude extract containing the glyphosate tolerance-conferring protein with glyphosate, and assaying for the products of glyphosate degradation. Examples of methods to measure such activity in both GDC-1 and GDC-2 are provided in the Example section.

Herbicide Resistance Proteins

Preferred herbicide resistance proteins for use in the methods of the present invention are decarboxylase enzymes. Examples of decarboxylase enzymes that may be used are provided in Table 1. In one embodiment the GDC-1 coding sequence, as disclosed in co-pending U.S. Application entitled “GDC-1 Genes Conferring Herbicide Resistance”, filed concurrently herewith, is the herbicide resistance protein. In another embodiment, the GDC-2 coding sequence, as disclosed in co-pending U.S. Application entitled “GDC-2 Genes Conferring Herbicide Resistance”, filed concurrently herewith, is the herbicide resistance protein.

Methods of the invention also encompass variant nucleic acid molecules that are sufficiently identical to the sequences provided for representative decarboxylase enzymes. “Variants” of the herbicide resistance-encoding nucleotide sequences include those sequences that encode the decarboxylase proteins disclosed herein but that differ conservatively because of the degeneracy of the genetic code, as well as those that are sufficiently identical as described below. Naturally occurring allelic variants can be identified with the use of well-known molecular biology techniques, such as polymerase chain reaction (PCR) and hybridization techniques as outlined below. Variant nucleotide sequences also include synthetically derived nucleotide sequences that have been generated, for example, by using site-directed mutagenesis but which still encode the decarboxylase proteins disclosed in the present invention as discussed below. Variant proteins for use in the methods of the present invention are biologically active, that is they retain the desired biological activity of the native protein, that is, herbicide resistance activity. By “retains herbicide resistance activity” is intended that the variant will have at least about 30%, preferably at least about 50%, more preferably at least about 70%, even more preferably at least about 80% of the herbicide resistance activity of the native protein. Methods for measuring herbicide resistance activity are well known in the art. See, for example, U.S. Pat. Nos. 4,535,060, and 5,188,642, each of which are herein incorporated by reference in their entirety.

The term “sufficiently identical” is intended an amino acid or nucleotide sequence that has at least about 60% or 65% sequence identity, preferably about 70% or 75% sequence identity, more preferably about 80% or 85% sequence identity, most preferably about 90%, 91%, 92%, 93%, 94%, 95%, 96%, 97%, 98% or 99% sequence identity compared to a reference sequence using one of the alignment programs described herein using standard parameters. One of skill in the art will recognize that these values can be appropriately adjusted to determine corresponding identity of proteins encoded by two nucleotide sequences by taking into account codon degeneracy, amino acid similarity, reading frame positioning, and the like.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLASTN and BLASTX programs of Altschul et al. (1990) J. Mol. Biol. 215:403. BLAST nucleotide searches can be performed with the BLASTN program, score=100, wordlength=12, to obtain nucleotide sequences homologous to herbicide resistance-encoding nucleic acid molecules used in methods of the invention. BLAST protein searches can be performed with the BLASTX program, score=50, wordlength=3, to obtain amino acid sequences homologous to herbicide resistance protein molecules expressed using the methods of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. See www.ncbi.nlm.nih.gov. Another non-limiting example of a mathematical algorithm utilized for the comparison of sequences is the ClustalW algorithm (Higgins et al. (1994). Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the vector NTi Program Suite (Informax, Inc). After alignment of amino acid sequences with ClustalW, the percent amino acid identity can be assessed. A non-limiting example of a software program useful for analysis of ClustalW alignments is GeneDoc™. Genedoc™ (Karl Nicholas) allows assessment of amino acid (or DNA) similarity and identify between multiple proteins. Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package (available from Accelrys, Inc., 9865 Scranton Rd., San Diego, Calif., USA). When utilizing the ALIGN program for comparing amino acid sequences, a PAM120 weight residue table, a gap length penalty of 12, and a gap penalty of 4 can be used.

A preferred program is GAP version 10, which used the algorithm of Needleman and Wunsch (1970) supra. GAP Version 10 may be used with the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 Scoring Matrix. Equivalent programs may also be used. By “equivalent program” is intended any sequence comparison program that, for any two sequences in question, generates an alignment having identical nucleotide or amino acid residue matches and an identical percent sequence identity when compared to the corresponding alignment generated by GAP Version 10.

The skilled artisan will further appreciate that changes can be introduced by mutation into the nucleotide sequences used in the methods of the invention, thereby leading to changes in the amino acid sequence of the encoded herbicide resistance proteins, without altering the biological activity of the proteins. Thus, variant isolated nucleic acid molecules can be created by introducing one or more nucleotide substitutions, additions, or deletions into the corresponding nucleotide sequence disclosed herein, such that one or more amino acid substitutions, additions or deletions are introduced into the encoded protein. Mutations can be introduced by standard techniques, such as site-directed mutagenesis and PCR-mediated mutagenesis. Methods using such variant nucleotide sequences are also encompassed by the present invention.

For example, preferably, conservative amino acid substitutions may be made at one or more predicted, preferably nonessential amino acid residues. A “nonessential” amino acid residue is a residue that can be altered from the wild-type sequence of an herbicide resistance protein without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Amino acid substitutions may be made in nonconserved regions that retain function. In general, such substitutions would not be made for conserved amino acid residues, or for amino acid residues residing within a conserved motif, where such residues are essential for protein activity. However, one of skill in the art would understand that functional variants may have minor conserved or nonconserved alterations in the conserved residues.

Alternatively, variant nucleotide sequences can be made by introducing mutations randomly along all or part of the coding sequence, such as by saturation mutagenesis, and the resultant mutants can be screened for ability to confer herbicide resistance activity to identify mutants that retain activity. Following mutagenesis, the encoded protein can be expressed recombinantly, and the activity of the protein can be determined using standard assay techniques.

The methods of the invention also encompass nucleic acid molecules comprising nucleotide sequences encoding partial-length herbicide resistance proteins. Nucleic acid molecules that are fragments of the herbicide resistance-encoding nucleotide sequences are also encompassed by the present invention. By “fragment” is intended a portion of the nucleotide sequence encoding an herbicide resistance protein. A fragment of a nucleotide sequence may encode a biologically active portion of an herbicide resistance protein, or it may be a fragment that can be used as a hybridization probe or PCR primer using methods disclosed below. Nucleic acid molecules that are fragments of an herbicide resistance nucleotide sequence comprise at least about 15, 20, 50, 75, 100, 200, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600 nucleotides, or up to the number of nucleotides present in a full-length herbicide resistance-encoding nucleotide sequence (for example, 2210 nucleotides for SEQ ID NO:1) depending upon the intended use. Fragments of the nucleotide sequences will encode protein fragments that retain the biological activity of the native herbicide resistance protein. By “retains herbicide resistance activity” is intended that the fragment will have at least about 30%, preferably at least about 50%, more preferably at least about 70%, even more preferably at least about 80% of the herbicide resistance activity of the native herbicide resistance protein. Methods for measuring herbicide resistance activity are well known in the art. See, for example, U.S. Pat. Nos. 4,535,060, and 5,188,642, each of which are herein incorporated by reference in their entirety.

A fragment of an herbicide resistance encoding nucleotide sequence that encodes a biologically active portion of a protein of the invention will encode at least about 15, 25, 30, 50, 75, 100, 125, 150, 175, 200, 250, 300, 350, 400, 450, 500, or 550 contiguous amino acids, or up to the total number of amino acids present in a full-length herbicide resistance protein for use with methods of the invention (for example, 575 amino acids for SEQ ID NO: 3).

Altered or Improved Variants

It is recognized that DNA sequence of an herbicide resistance gene may be altered by various methods, and that these alterations may result in DNA sequences encoding proteins with amino acid sequences different that that encoded by an herbicide resistance gene. This protein may be altered in various ways including amino acid substitutions, deletions, truncations, and insertions. Methods for such manipulations are generally known in the art. For example, amino acid sequence variants of the herbicide resistance protein can be prepared by mutations in the DNA. This may also be accomplished by one of several forms of mutagenesis and/or in directed evolution. In some aspects, the changes encoded in the amino acid sequence will not substantially affecting function of the protein. Such variants will possess the desired herbicide resistance activity. However, it is understood that the ability of an herbicide resistance gene to confer herbicide resistance may be improved by one use of such techniques upon the compositions of this invention. For example, one may express an herbicide resistance gene in host cells that exhibit high rates of base misincorporation during DNA replication, such as XL-1 Red (Stratagene). After propagation in such strains, one can isolate the herbicide resistance DNA (for example by preparing plasmid DNA, or by amplifying by PCR and cloning the resulting PCR fragment into a vector), culture the herbicide resistance mutations in a non-mutagenic strain, and identify mutated herbicide resistance genes with improved resistance to herbicide, for example by growing cells in increasing concentrations of herbicide such as glyphosate, and testing for clones that confer an ability to tolerate increased concentrations of glyphosate.

Alternatively, alterations may be made to the protein sequence of many proteins at the amino or carboxy terminus without substantially affecting activity. This can include insertions, deletions, or alterations introduced by modern molecular methods, such as PCR, including PCR amplifications that alter or extend the protein coding sequence by virtue of inclusion of amino acid encoding sequences in the oligonucleotides utilized in the PCR amplification. Alternatively, the protein sequences added can include entire protein-coding sequences, such as those used commonly in the art to generate protein fusions. Such fusion proteins are often used to (1) increase expression of a protein of interest (2) introduce a binding domain, enzymatic activity, or epitope to facilitate either protein purification, protein detection, or other experimental uses known in the art (3) target secretion or translation of a protein to a subcellular organelle, such as the periplasmic space of Gram-negative bacteria, or the endoplasmic reticulum of eukaryotic cells, the latter of which often results in glycosylation of the protein.

Variant nucleotide and amino acid sequences of the present invention also encompass sequences derived from mutagenic and recombinogenic procedures such as DNA shuffling. With such a procedure, one or more different herbicide resistance protein coding regions can be used to create a new herbicide resistance protein possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the herbicide resistance gene of the invention and other known herbicide resistance genes to obtain a new gene coding for a protein with an improved property of interest, such as an increased glyphosate resistance activity. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

TABLE 1
Listing of enzyme classes in Expasy database listing TPP as a cofactor

| | | Cofactors other | Exemplary |
|---|---|---|---|
| EC | | than TPP (if | GENBANK |
| Number | Enzyme Classification | any) | Accession No. |
| 1.2.1.58 | Phenylglyoxylate dehydrogenase (acylating) | FAD | AJ428571 |
| 1.2.2.2 | Pyruvate dehydrogenase (cytochrome) | | AAC73958 |
| 1.2.3.3 | Pyruvate oxidase | FAD | X04105 |
| | | | L39074 |
| 1.2.4.1 | Pyruvate dehydrogenase (lipoamide) | | U09865 |
| 1.2.4.2 | Oxoglutarate dehydrogenase (lipoamide) | | X91877 |
| 1.2.4.4 | 3-methyl-2-oxobutanoate dehydrogenase | | M97391 |
| | (lipoamide) | | |
| 2.2.1.1 | Transketolase | | Z73234 |
| 2.2.1.3 | Formaldehyde transketolase | | X02424 |
| 2.2.1.4 | Acetoin—ribose-5-phosphate transaldolase | | ND |
| 4.1.1.1 | Pyruvate decarboxylase | | U00967 |
| 4.1.1.7 | Benzoylformate decarboxylase | | J05293 |
| 4.1.1.8 | Oxalyl-CoA decarboxylase | | M77128 |
| 4.1.1.47 | Tartronate-semialdehyde synthase | | L03845 |
| 4.1.1.71 | 2-oxoglutarate decarboxylase | | M21787 |
| 4.1.1.74 | Indolepyruvate decarboxylase | Mg++ | L26240 |
| | | | D90214 |
| 4.1.1.75 | 5-guanidino-2-oxopentanoate decarboxylase | Divalent Cation | ND |
| 4.1.2.9 | Phosphoketolase | | AJ309011 |
| 4.1.2.22 | Fructose-6-phosphate phosphoketolase | | AJ293946 |
| 4.1.2.38 | Benzoin aldolase | | U04048 |
| 4.1.3.15 | 2-hydroxy-3-oxoadipate synthase | | ND |
| 4.1.3.18 | Acetolactate synthase | | L04470 |
| 4.1.3.37 | 1-deoxy-D-xylulose 5-phosphate synthase | | AF035440 |
| 4.4.1.12 | Sulfoacetaldehyde lyase | | AF305552 |

The sequences obtained through the Genbank accession numbers are herein incorporated by reference in their entirety.
Methods of Identifying/Isolating Herbicide Resistance Genes

Herbicide resistance genes may be identified by isolating DNA or cDNA from an organism, preferably an organism that is capable of growing in herbicidal or antibiotic concentrations of an herbicide. A library of clones (DNA or cDNA clones) can be transformed into a test organism, such as a bacterium. For example, E. coli may function as a test organism. The individual clones can be then grown on media containing the herbicide or antibiotic, at a concentration at which the test organism does not grow, or grows noticeably slower or to a noticeably lower density than cells grown in media lacking the herbicide. The clones conferring tolerance of the test cells to the herbicide (“positive clones”) can then be identified. The DNA sequences of the positive clones are analyzed, and compared to databases of known proteins such as the Genbank ‘nr’ database. Finally, those positive clones with homology to known decarboxylases, or minimally having amino acid homology to a TPP-binding domain, can be identified.

Alternatively, sets of DNA sequences of genes or gene fragments may be screened, such as the Genbank database, or the Genbank EST database, and genes likely to encode decarboxylases or likely to have TPP-binding domains may be identified. Then, the genes could be cloned into a vector in such a way that the gene is expressed in a test cell, such as an E. coli cell. Finally, the cells expressing the genes could be tested at various concentrations of an herbicide, and those conferring resistance to an herbicide, such as glyphosate, could be identified.

A known sequence of a TPP-binding protein may be used to generate DNA probes. Then these DNA probes can be utilized to screen a library (libraries) composed of cloned DNA, or cloned cDNA from one or more organisms by methods known in the art for identifying homologous gene sequences. The homologous genes (if needed) can be engineered to be expressed in a test cell (such as an E. coli cell). Clones conferring increased tolerance to an herbicide may be identified and sequenced.

Alternatively, proteins having TPP-binding characteristics may be purified, for example, by covalently attaching TPP to a solid matrix, such as a bead, and adsorbing crude or partially purified protein extracts to the bead, washing the bead, and eluting the TPP-binding protein, for example by varying salt, pH, or other conditions that cause the TPP molecule to no longer bind the TPP-binding domain. The protein purified in this way can identify gene(s) likely to have herbicide resistance properties by obtaining a partial amino acid sequence of the protein, for example by performing amino-terminal amino acid sequencing. Upon knowing a sufficient portion of the amino acid sequence, the gene encoding this protein may be cloned by methods known in the art.

Genes containing such TPP-binding domains can also be identified directly, for example by phage display or cell surface display technologies. Phage display methods are based on expressing recombinant proteins or peptides fused to a phage coat protein. Such phage are then used to perform binding assays, and phage containing inserts conferring binding ability (such as by expression of a TPP-binding domain) are retained, and can be propagated using traditional phage bacteriology techniques. Bacterial display is a modification of phage display based on expressing recombinant proteins fused to sorting signals that direct their incorporation on the cell surface. Methods for phage display and bacterial display are well known in the art. For example, see Benhar (2001) Biotechnol. Adv. 19:1-33, or Hartley (2002) J. Recept. Signal Transduct. Res. 22:373-92, and references within.

In addition, having provided that TPP-binding proteins are capable of conferring herbicide resistance, and it being understood that many TPP-binding proteins are known to exist, and that additional TPP-binding enzymes may be identified by virtue of their amino acid homology, additional herbicide-resistance encoding proteins may be identified by testing one or all of the subset of known TPP-binding proteins by one or all of the assays described, in order to assess the herbicide resistance-conferring ability of the protein.

Alternatively, the DNA sequence of any of the known classes of TPP-binding proteins may be used to identify novel related proteins, which are also likely to bind TPP as a consequence of their catalytic role. Thus, having identified TPP-binding proteins by this way, the herbicide resistance conferring ability of such genes may be assessed.

Additionally, corresponding herbicide resistance sequences can be identified by using methods such as PCR, hybridization, and the like. See, for example, Sambrook J., and Russell, D. W. (2001) Molecular Cloning: A Laboratory Manual. (Cold Spring Harbor Laboratory Press, Cold Spring Harbor, N.Y.) and Innis, et al. (1990) PCR Protocols: A Guide to Methods and Applications (Academic Press, NY).

In a hybridization method, all or part of the herbicide resistance nucleotide sequence can be used to screen cDNA or genomic libraries. Methods for construction of such cDNA and genomic libraries are generally known in the art and are disclosed in Sambrook and Russell, 2001. See also Sambrook et al. (1989) Molecular Cloning: A Laboratory Manual (2d ed., Cold Spring Harbor Laboratory Press, Plainview, N.Y.). The so-called hybridization probes may be genomic DNA fragments, cDNA fragments, RNA fragments, or other oligonucleotides, and may be labeled with a detectable group such as ³²P, or any other detectable marker, such as other radioisotopes, a fluorescent compound, an enzyme, or an enzyme co-factor. Probes for hybridization can be made by labeling synthetic oligonucleotides based on the known herbicide resistance-encoding nucleotide sequence disclosed herein. Degenerate primers designed on the basis of conserved nucleotides or amino acid residues in the nucleotide sequence or encoded amino acid sequence can additionally be used. The probe typically comprises a region of nucleotide sequence that hybridizes under stringent conditions to at least about 12, preferably about 25, more preferably about 50, 75, 100, 125, 150, 175, 200, 250, 300, 350, or 400 consecutive nucleotides of herbicide resistance encoding nucleotide disclosed herein or a fragment or variant thereof. Preparation of probes for hybridization is generally known in the art and is disclosed in Sambrook and Russell, 2001, herein incorporated by reference.

For example, an entire herbicide resistance sequence disclosed herein, or one or more portions thereof, may be used as a probe capable of specifically hybridizing to corresponding herbicide resistance sequences and messenger RNAs. To achieve specific hybridization under a variety of conditions, such probes include sequences that are unique and are preferably at least about 10 nucleotides in length, and most preferably at least about 20 nucleotides in length. Such probes may be used to amplify corresponding herbicide resistance sequences from a chosen organism by PCR. This technique may be used to isolate additional coding sequences from a desired organism or as a diagnostic assay to determine the presence of coding sequences in an organism. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours.

Specificity is typically the function of post-hybridization washes, the critical factors being the ionic strength and temperature of the final wash solution. For DNA—DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

Transformation of Cells

Transformation of bacterial cells is accomplished by one of several techniques known in the art, not limited to electroporation, or chemical transformation (see for example Ausubel (ed.), Current Protocols in Molecular Biology, John Wiley and Sons, Inc. (1994)). Markers conferring resistance to toxic substances are useful in identifying transformed cells (having taken up and expressed the test DNA) from non-transformed cells (those not containing or not expressing the test DNA). By engineering the herbicide resistance gene to be (1) expressed from a bacterial promoter known to stimulate transcription in the organism to be tested, (2) properly translated to generate an intact herbicide resistance peptide, and (3) placing the cells in an otherwise toxic concentration of herbicide, one can identify cells that have been transformed with DNA by virtue of their resistance to herbicide.

Transformation of plant cells can be accomplished in similar fashion. First, one engineers the herbicide resistance gene in a way that allows its expression in plant cells. The organization of such constructs is well known in the art.

The herbicide resistance sequences used in the methods of the invention may be provided in expression cassettes for expression in the plant of interest. The cassette will include 5′ and 3′ regulatory sequences operably linked to a sequence of the invention. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

Such an expression cassette is provided with a plurality of restriction sites for insertion of the herbicide resistance sequence to be under the transcriptional regulation of the regulatory regions.

The expression cassette will include in the 5′-3′ direction of transcription, a transcriptional and translational initiation region (i.e., a promoter), a DNA sequence of the invention, and a transcriptional and translational termination region (i.e., termination region) functional in plants. The promoter may be native or analogous, or foreign or heterologous, to the plant host and/or to the DNA sequence of the invention. Additionally, the promoter may be the natural sequence or alternatively a synthetic sequence. Where the promoter is “native” or “homologous” to the plant host, it is intended that the promoter is found in the native plant into which the promoter is introduced. Where the promoter is “foreign” or “heterologous” to the DNA sequence of the invention, it is intended that the promoter is not the native or naturally occurring promoter for the operably linked DNA sequence of the invention.

The termination region may be native with the transcriptional initiation region, may be native with the operably linked DNA sequence of interest, may be native with the plant host, or may be derived from another source (i.e., foreign or heterologous to the promoter, the DNA sequence of interest, the plant host, or any combination thereof). Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Where appropriate, the gene(s) may be optimized for increased expression in the transformed host cell. That is, the genes can be synthesized using host cell-preferred codons for improved expression, or may be synthesized using codons at a host-preferred codon usage frequency. Generally, the GC content of the gene will be increased. See, for example, Campbell and Gowri (1990) Plant Physiol. 92:1-11 for a discussion of host-preferred codon usage. Methods are known in the art for synthesizing host-preferred genes. See, for example, U.S. Pat. Nos. 6,320,100; 6,075,185; 5,380,831; and 5,436,391, U.S. Published Application Nos. 20040005600 and 20010003849, and Murray et al. (1989) Nucleic Acids Res. 17:477-498, herein incorporated by reference.

In some instances, it may be useful to engineer the gene such that the resulting peptide is secreted, or otherwise targeted within the plant cell. For example, the gene can be engineered to contain a signal peptide to facilitate transfer of the peptide to the endoplasmic reticulum. It may also be preferable to engineer the plant expression cassette to contain an intron, such that mRNA processing of the intron is required for expression. In one embodiment, the nucleic acids of interest are targeted to the chloroplast for expression. In this manner, where the nucleic acid of interest is not directly inserted into the chloroplast, the expression cassette will additionally contain a nucleic acid encoding a transit peptide to direct the gene product of interest to the chloroplasts. Such transit peptides are known in the art. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

Chloroplast targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl)shikimate-3-phosphate synthase (EPSPS) (Archer et al. (1990) J. Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); and the light harvesting chlorophyll a/b binding protein (LHBP) (Lamppa et al. (1988) J. Biol. Chem. 263:14996-14999). See also Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

Methods for transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

The nucleic acids of interest to be targeted to the chloroplast may be optimized for expression in the chloroplast to account for differences in codon usage between the plant nucleus and this organelle. In this manner, the nucleic acids of interest may be synthesized using chloroplast-preferred codons. See, for example, U.S. Pat. No. 5,380,831, herein incorporated by reference.

Typically this ‘plant expression cassette’ will be inserted into a ‘plant transformation vector’. This plant transformation vector may be comprised of one or more DNA vectors needed for achieving plant transformation. For example, it is a common practice in the art to utilize plant transformation vectors that are comprised of more than one contiguous DNA segment. These vectors are often referred to in the art as ‘binary vectors’. Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a ‘gene of interest’ (a gene engineered to be capable of expression in a plant cell for which generation of transgenic plants is desired). Also present on this plasmid vector are sequences required for bacterial replication. The cis-acting sequences are arranged in a fashion to allow efficient transfer into plant cells and expression therein. For example, the selectable marker gene and the gene of interest are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as in understood in the art (Hellens and Mullineaux (2000) Trends in Plant Science 5:446-451). Several types of Agrobacterium strains (e.g. LBA4404, GV3101, EHA101, EHA105, etc.) can be used for plant transformation. The second plasmid vector is not necessary for transforming the plants by other methods such as microprojection, microinjection, electroporation, polyethelene glycol, etc. Many types of vectors can be used to transform plant cells for achieving herbicide resistance.

In general, plant transformation methods involve transferring heterologous DNA into target plant cells (e.g. immature or mature embryos, suspension cultures, undifferentiated callus, protoplasts, etc.), followed by applying a maximum threshold level of appropropriate selection (depending on the selectable marker gene and in this case “glyphosate”) to recover the transformed plant cells from a group of untransformed cell mass. Explants are typically transferred to a fresh supply of the same medium and cultured routinely. Subsequently, the transformed cells are differentiated into shoots after placing on regeneration medium supplemented with a maximum threshold level of selecting agent (e.g. “glyphosate”). The shoots are then transferred to a selective rooting medium for recovering rooted shoot or plantlet. The transgenic plantlet then grow into mature plant and produce fertile seeds (e.g. Hiei et al. (1994) The Plant Journal 6:271-282; Ishida et al. (1996) Nature Biotechnology 14:745-750). Explants are typically transferred to a fresh supply of the same medium and cultured routinely. A general description of the techniques and methods for generating transgenic plantlets are found in Ayres and Park (1994) Critical Reviews in Plant Science 13:219-239, and Bommineni and Jauhar (1997) Maydica 42:107-120. Since the transformed material contains many cells; both transformed and non-transformed cells are present in any piece of subjected target callus or tissue or group of cells. The ability to kill non-transformed cells and allow transformed cells to proliferate results in transformed plant cultures. Often, the ability to remove non-transformed cells is a limitation to rapid recovery of transformed plant cells and successful generation of transgenic plants.

Generation of transgenic plants may be performed by one of several methods, including but not limited to introduction of heterologous DNA by Agrobacterium into plant cells (Agrobacterium-mediated transformation), bombardment of plant cells with heterologous foreign DNA adhered to particles including aerosol beam transformation (U.S. Published Application No. 20010026941; U.S. Pat. No. 4,945,050; International Publication No. WO 91/00915; U.S. Published Application No. 2002015066), and various other non-particle direct-mediated methods (e.g. Hiei et al. (1994) The Plant Journal 6: 271-282; Ishida et al. (1996) Nature Biotechnology 14: 745-750; Ayres and Park (1994) Critical Reviews in Plant Science 13: 219-239; Bommineni and Jauhar (1997) Maydica 42: 107-120) to transfer DNA.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

Following integration of heterologous foreign DNA into plant cells, one then applies a maximum threshold level of herbicide in the medium to kill the untransformed cells and separate and proliferate the putatively transformed cells that survive from this selection treatment by transferring regularly to a fresh medium. By continuous passage and challenge with herbicide, one identifies and proliferates the cells that are transformed with the plasmid vector. Then molecular and biochemical methods will be used for confirming the presence of the integrated heterologous gene of interest in the genome of transgenic plant.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

Evaluation of Plant Transformation

Following introduction of heterologous foreign DNA into plant cells, the transformation or integration of heterologous gene in the plant genome is confirmed by various methods such as analysis of nucleic acids, proteins and metabolites associated with the integrated gene.

PCR Analysis: PCR analysis is a rapid method to screen transformed cells, tissue or shoots for the presence of incorporated gene at the earlier stage before transplanting into the soil (Sambrook and Russell, 2001). PCR is carried out using oligonucleotide primers specific to the gene of interest or Agrobacterium vector background, etc.

Southern Analysis: Plant transformation is confirmed by Southern blot analysis of genomic DNA (Sambrook and Russell, 2001). In general, total DNA is extracted from the transformant, digested with appropriate restriction enzymes, fractionated in an agarose gel and transferred to a nitrocellulose or nylon membrane. The membrane or “blot” then is probed with, for example, radiolabeled ³²P target DNA fragment to confirm the integration of introduced gene in the plant genome according to standard techniques (Sambrook and Russell, 2001. Molecular Cloning: A Laboratory Manual. Cold Spring Harbor Laboratory Press, Cold Spring Harbor, N.Y.

Northern Analysis: RNA is isolated from specific tissues of transformant, fractionated in a formaldehyde agarose gel, blotted onto a nylon filter according to standard procedures that are routinely used in the art (Sambrook, J., and Russell, D. W. 2001. Molecular Cloning: A Laboratory Manual, Cold Spring Harbor Laboratory Press, Cold Spring Harbor, N.Y.) Expression of RNA encoded by the herbicide resistance gene is then tested by hybridizing the filter to a radioactive probe derived from an herbicide resistance gene, by methods known in the art (Sambrook and Russell, 2001).

Western blot and Biochemical assays: Western blot and biochemical assays and the like may be carried out on the transgenic plants to confirm the determine the presence of protein encoded by the herbicide resistance gene by standard procedures (Sambrook, J., and Russell, D. W. 2001. Molecular Cloning: A Laboratory Manual, Cold Spring Harbor Laboratory Press, Cold Spring Harbor, N.Y.) using antibodies that bind to one or more epitopes present on the herbicide resistance protein.

Herbicide Resistant Plants

In another aspect of the invention, one may generate transgenic plants expressing an herbicide resistance gene that are more resistant to high concentrations of herbicide than non-transformed plants. Methods described above by way of example may be utilized to generate transgenic plants, but the manner in which the transgenic plant cells are generated is not critical to this invention. Methods known or described in the art such as Agrobacterium-mediated transformation, biolistic transformation, and non-particle-mediated methods may be used at the discretion of the experimenter. Plants expressing an herbicide resistance gene may be isolated by common methods described in the art, for example by transformation of callus, selection of transformed callus, and regeneration of fertile plants from such transgenic callus. In such process, an herbicide resistance gene may be used as selectable marker. Alternatively, one may use any gene as a selectable marker so long as its expression in plant cells confers ability to identify or select for transformed cells. Genes known to function effectively as selectable markers in plant transformation are well known in the art.

The following examples are offered by way of illustration and not by way of limitation.

### EXPERIMENTAL

### EXAMPLE 1

### GDC-1 and GDC-2 Confer Glyphosate Resistance Upon Cells

Starter cultures of E. coli containing GDC-1(full), GDC-2, or vector alone were grown overnight in LB media, diluted 1:1000 into 3 ml M9 minimal media containing 0, 2, 5, 10, 20 and 30 mM glyphosate and grown at 37° C. Each strain was grown in triplicate at each concentration. OD₆₀₀ was measured at 0, 7, 24, and 28 hours after inoculation. Table 2 shows the OD₆₀₀ obtained for each construct at 28 hours after inoculation.

TABLE 2
Growth of clones in glyphosate

| Glyphosate | Vector | | GDC-1 | | GDC-2 | |
|---|---|---|---|---|---|---|
| concentration | Mean | S.D | Mean | S.D. | Mean | S.D. |
| 0 | 0.052 | 0.001 | 0.049 | 0.006 | 0.050 | 0.001 |
| 2 | 0.038 | 0.001 | 0.056 | 0.000 | 0.054 | 0.001 |
| 5 | 0.038 | 0.001 | 0.055 | 0.001 | 0.056 | 0.001 |
| 10 | 0.038 | 0.000 | 0.057 | 0.001 | 0.056 | 0.001 |
| 20 | 0.038 | 0.000 | 0.058 | 0.001 | 0.058 | 0.001 |

### EXAMPLE 2

### GDC-1 and GDC-2 are Both TPP-binding Decarboxylases

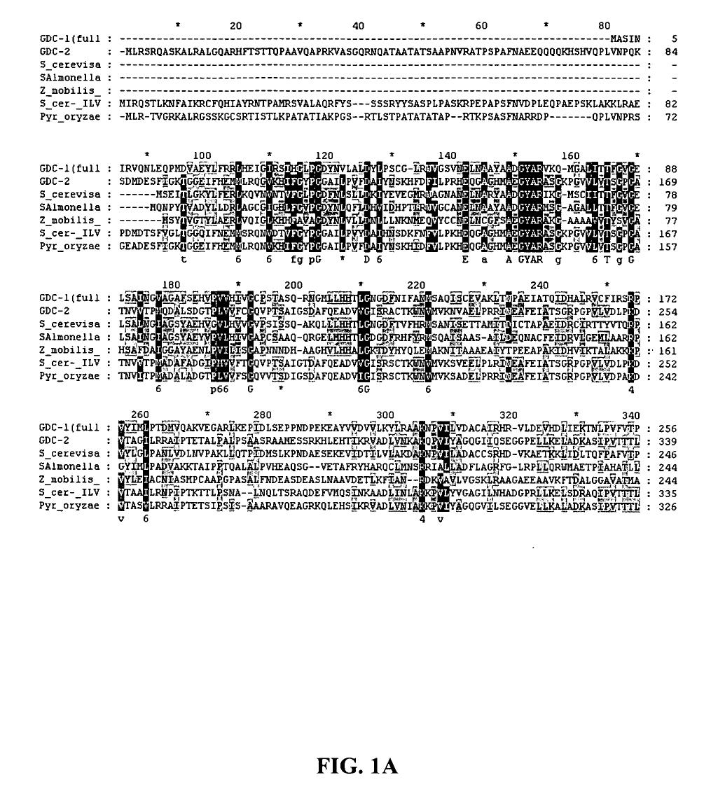

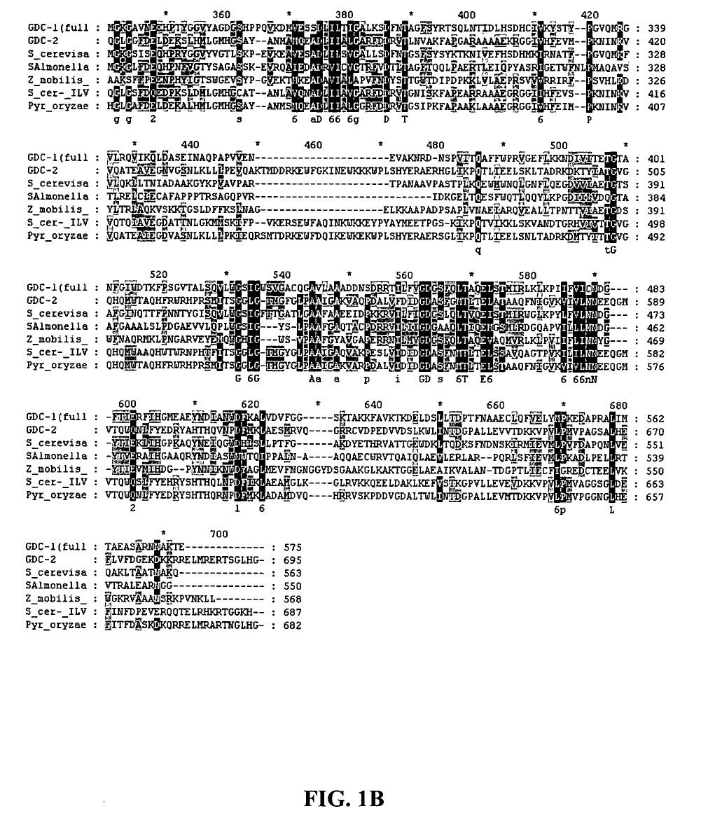

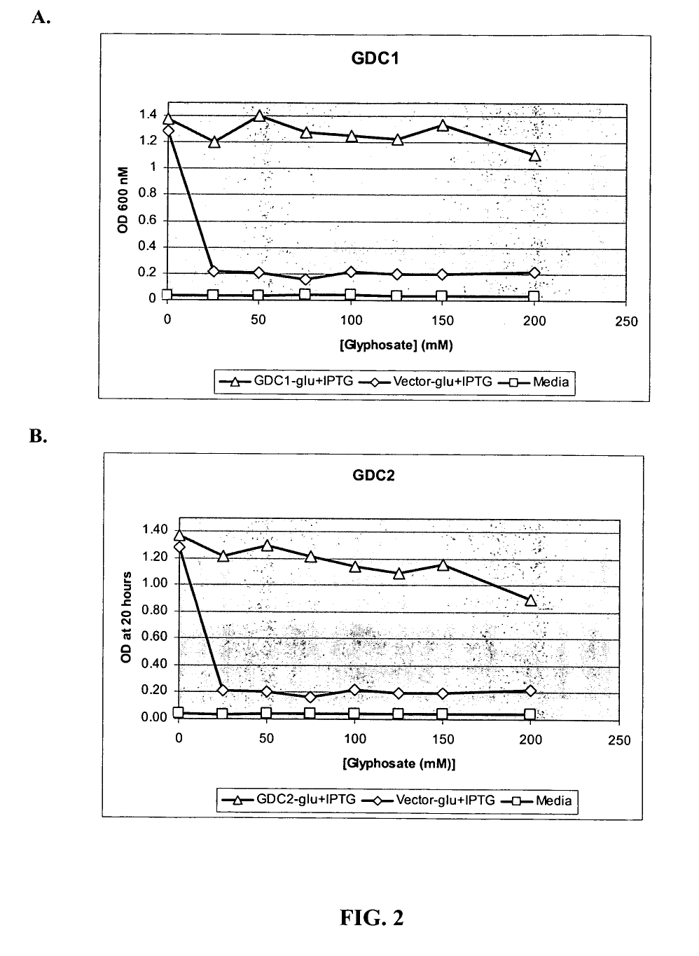

Searches of DNA and protein sequence databases, as well as sequence analysis of the GDC-1 and GDC-2 proteins show that they are homologous to pyruvate decarboxylase and acetolactate synthases. See, respectively, co-pending U.S. Application entitled “GDC-1 Genes Conferring Herbicide Resistance”, and co-pending U.S. Application entitled “GDC-2 Genes Conferring Herbicide Resistance”, both filed concurrently herewith. These searches reveal that both both GDC-1 and GDC-2 contain amino acid regions which are conserved among TPP-binding proteins, including pyruvate decarboxylases and acetolactate synthases. An alignment of GDC-1 and GDC-2 with other known TPP-binding proteins is shown in FIG. 1.

### EXAMPLE 3

### Engineering GDC-1 and GDC-2 for Expression in E. coli

E. coli strains expressing GDC-1 and GDC-2 were engineered into a customized expression vector, pAX481. pAX481 contains the pBR322 origin of replication, a chloramphenicol acetyl transferase gene (for selection and maintenance of the plasmid), the lacI gene, the Ptac promoter and the rrnB transcriptional terminator. The GDC-1 and GDC-2 open reading frames were amplified by PCR, using a high fidelity DNA polymerase, as known in the art. The oligonucleotides for PCR amplification of GDC-1 and GDC-2 were designed to place the ATG start site of the genes at the proper distance from the ribosome binding site of pAX481.

The GDC-1 PCR products were cloned into the expression vector pAX481 and transformed into E. coli XL1 Blue MRF′ to yield the plasmid pAX472. The GDC-2 PCR product was cloned into the expression vector pAX481 and transformed into E. coli XL1 Blue MRF′ to yield the plasmid pAX473. Postive clones were identified by standard methods known in the art. The sequences of pAX472 and pAX473 were confirmed by DNA sequence analysis as known in the art.

### EXAMPLE 4

### GDC-1 and GDC-2 Confer Resistance to High Levels of Glyphosate

E. coli strains containing either GDC-1 (pAX472) or GDC-2 (pAX473) expression vectors, or vector controls (pAX481), were grown to saturation in M63 media, and diluted into a 48-well plate by adding 40 μl of cells to 1 ml cultures. Cultures contained M63 (13.6 g KH₂PO₄; 2 g (NH₄)₂SO₄; 0.5 mg FeSO₄-7H₂O; 2.4 mg MgCl₂ in 1 liter dH₂O) supplemented with proline and thiamine, 20 ug/ml chloramphenicol, 0.5% glucose, and from 0 to 200 mM glyphosate, diluted from a 1 M stock solution. 1 mM IPTG was added to each well to induce protein expression.

Cultures were grown at 37° C. with shaking at 300 rpm. At 0 hours and at 42 hours, 300 ml of culture was withdrawn and placed into a 96-well assay plate. The absorbance of the culture at 600 nm was measured in a 96-well plate using a Spectramax 190 Spectrophotometer (Molecular Devices, Inc.). The absorbance of the cultures at 0 hours was consistently below 0.04. The table below shows the absorbance at 600 nM obtained from the individual cultures after 42 hours of incubation.

TABLE 3
GDC 1 and GDC-2 confer glyphosate
resistance upon sensitive cells

| [Gly] mM | GDC1 | GDC2 | Vector | Media |
|---|---|---|---|---|
| 0 | 1.37 | 1.37 | 1.28 | 0.04 |
| 25 | 1.20 | 1.21 | 0.21 | 0.04 |
| 50 | 1.40 | 1.30 | 0.21 | 0.04 |
| 75 | 1.27 | 1.22 | 0.16 | 0.04 |
| 100 | 1.26 | 1.14 | 0.22 | 0.04 |
| 125 | 1.23 | 1.09 | 0.20 | 0.04 |
| 150 | 1.33 | 1.16 | 0.20 | 0.04 |
| 200 | 1.11 | 0.90 | 0.22 | 0.04 |

### EXAMPLE 5

### GDC-1 and GDC-2 do not Complement an aroA Mutation in E. coli

The E. coli aroA gene codes for EPSP synthase, the target enzyme for glyphosate. EPSP synthase catalyzes the sixth step in the biosynthesis of aromatic amino acids in microbes and plants. aroA mutants that lack an EPSP synthase do not grow on minimal media that lacks aromatic amino acids (Pittard and Wallace (1966) J. Bacteriol. 91:1494-508), but can grow in rich media, such as LB. However, genes encoding EPSPS activity can restore ability to grow on glyphosate upon aroA mutant E. coli strains. Thus, a test for genetic complementation of an aroA mutant is a highly sensitive method to test if a gene is capable of functioning as an EPSPS in E. coli. Such tests for gene function by genetic complementation are known in the art.

A deletion of the aroA gene was created in E. coli XL-1 MRF′ (Stratagene) by PCR/recombination methods known in the art and outlined by Datsenko and Wanner, (2000) Proc. Natl. Acad. Sci. USA 97:6640-6645. This system is based on the Red system that allows for chromosomal disruptions of targeted sequences. A large portion (1067 nt of the 1283 nt) of the aroA coding region was disrupted by the engineered deletion. The presence of the deletion was confirmed by PCR with several sets of oligonucleotides, and by the appearance of an aroA phenotype in the strain, referred to herein as ‘ΔaroA’. ΔaroA grows on LB media (which contains all amino acids) and grows on M63 media supplemented with phenylalanine, tryptophan, and tyrosine, but does not grow on M63 minimal media (which lacks aromatic amino acids). These results indicate that ΔaroA exhibits an aroA phenotype.

The ability of an EPSPS to complement the mutant phenotype of ΔaroA was confirmed. Clone pAX482, an E. coli expression vector containing the wild-type E. coli aroA gene, was transformed into ΔaroA, and transformed cells were selected. These cells (containing a functional aroA gene residing on a plasmid) were then plated on LB media, M63, and M63 with amino acid supplements. Where the ΔaroA mutant strain grew only on LB and M63 supplemented with aromatic amino acids, ΔaroA cells containing the functional aroA gene on a plasmid grew on all three media types.

In order to determine if GDC-1 or GDC-2 could confer complementation, plasmid pAX472, the expression vector containing GDC-1, and pAX473, the expression vector containing GDC-2 were transformed into ΔaroA and plated on the same three types of media. Cells transformed with either pAX472 or pAX473 were able to grow on M63 media supplemented with phenylalanine, tryptophan, and tyrosine and LB media but they were not able to grow on M63 alone. Thus, neither GDC-1 nor GDC-2 are capable of complementing the aroA mutation, and thus neither GDC-1 nor GDC-2 is an EPSP synthase.

### EXAMPLE 6

### Purification of GDC-1 Expressed as a 6×His-tagged Protein in E. coli

The GDC-1 coding region (1,728 nucleotides) was amplified by PCR using ProofStart™ DNA polymerase. Oligonucleotides used to prime PCR were designed to introduce restriction enzyme recognition sites near the 5′ and 3′ ends of the resulting PCR product. The resulting PCR product was digested with BamH I and Sal I. BamH I cleaved the PCR product at the 5′ end, and Sal I cleaved the PCR product at the 3′ end. The digested product was cloned into the 6×His-tag expression vector pQE-30 (Qiagen), prepared by digestion with BamH I and Sal I. The resulting clone, pAX623, contained GDC-1 in the same translational reading frame as, and immediately C-terminal to, the 6×His tag of pQE-30. General strategies for generating such clones, and for expressing proteins containing 6×His-tag are well known in the art.

The ability of this clone to confer glyphosate resistance was confirmed by plating cells of pAX623 onto M63 media containing 5 mM glyphosate. pAX623 containing cells gave rise to colonies, where cells containing the vector alone gave no colonies.

GDC-1 protein from pAX623-containing cells was isolated by expression of GDC-1-6×His-tagged protein in E. coli, and the resulting protein purified using Ni-NTA Superflow Resin (Qiagen) as per manufacturer's instructions.

### EXAMPLE 7

### Assay of GDC-1 Pyruvate Decarboxylase Activity

100 ng of GDC-1 protein was tested for activity in a standard pyruvate decarboxylase assay (Gounaris et al. (1971) J. of Biol. Chem. 246:1302-1309). This assay is a coupled reaction where in the first step the pyruvate decarboxylase (PDC) converts pyruvate to acetaldehyde and CO₂. The acetaldehyde produced in this reaction is a substrate for alcohol dehydrogenase, which converts acetaldehyde and β-NADH to ethanol and β-NAD. Thus, PDC activity is detected by virtue of utilization of β-NADH as decrease in absorbance at 340 nM in a spectrophotometer. GDC-1 as well as a control enzyme (pyruvate decarboxylase, Sigma) were tested in this assay. GDC-1 showed activity as a pyruvate decarboxylase, and the reaction rate correlated with the concentration of pyruvate in the assay.

### EXAMPLE 8

### Assay of GDC-1 Ability to Modify Glyphosate

The ability of GDC-1 to modify glyphosate in vitro was tested by incubating GDC-1 with a mixture of radiolabeled and non-labeled glyphosate, and analyzing the reaction products by HPLC.

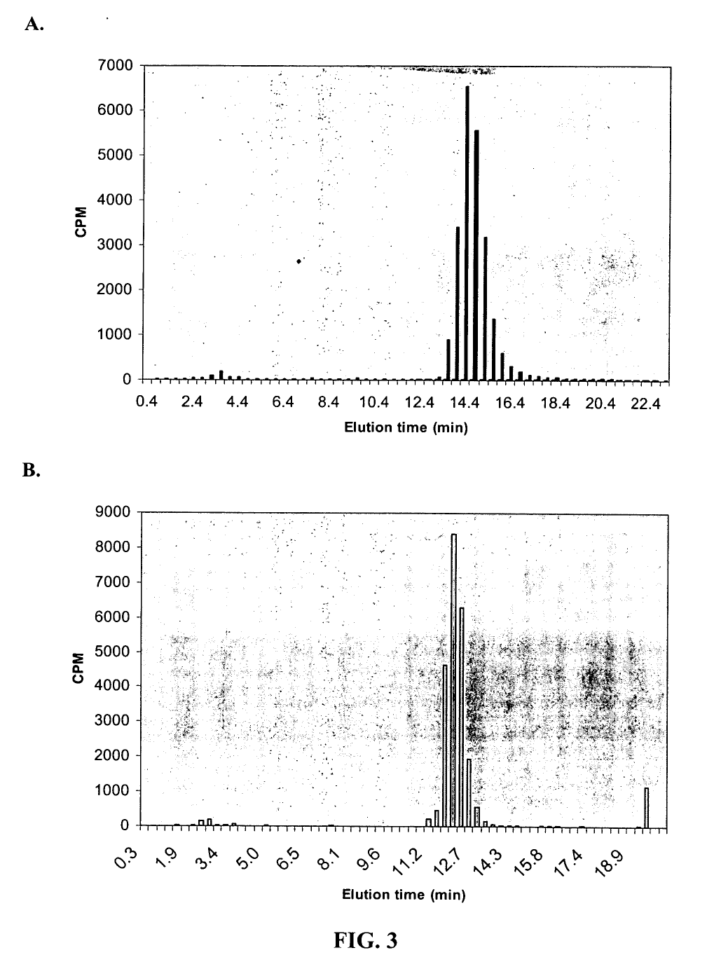

100 ng of GDC-1 purified protein was incubated with 20,000 cpm of C¹⁴ labeled glyphosate (NaOOCCH₂NH¹⁴CH₂PO₃H₂; Sigma catalog #G7014), mixed with unlabelled glyphosate to a final concentration of 2 mM in a reaction buffer of 200 mM Na-Citrate, pH 6.0, 1 mM TPP, 2 mM MgCl₂. Reaction was allowed to proceed 60 minutes, then 5 μl was applied to HPLC column (Dionex AminoPac PA10 analytical (and guard) column, anion exchange resin; Dionex Corporation). The column was equilibrated with 150 mM sodium hydroxide. Fractions were eluted with a sodium acetate gradient of 150-300 mM sodium acetate. Single drop (40 uL) fractions were collected, and the radioactivity present in each fraction determined using a 96-well scintillation counter. Analysis of the resulting data shows that GDC-1 converts a portion of the labeled glyphosate to a product with an elution time of approximately 19 minutes (FIG. 3B). Control experiments lacking purified GDC-1 show no peak at this elution time.

### EXAMPLE 9

### Purification of GDC-2 Expressed as a 6×His-tagged Protein in E. coli

The GDC-2 coding region (2,088 nucleotides) was amplified by PCR using ProofStart™ DNA polymerase (Qiagen). Oligonucleotides used to prime PCR were designed to introduce restriction enzyme recognition sites near the 5′ and 3′ ends of the resulting PCR product. The resulting PCR product was digested with BamH I and Hind III. BamH I cleaved the PCR product at the 5′ end, and Sal I cleaved the PCR product at the 3′ end. The digested product was cloned into the 6×His-tag expression vector pQE-30 (Qiagen), prepared by digestion with BamH I and Hind III. The resulting clone, pAX624, contained GDC-2 in the same translational reading frame as, and immediately C-terminal to, the 6×His tag of pQE-30. General strategies for generating such clones, and for expressing proteins containing 6×His-tag are well known in the art.

The ability of this clone to confer glyphosate resistance was confirmed by plating cells of pAX624 onto M63 media containing 5 mM glyphosate. pAX624 containing cells gave rise to colonies, where cells containing the vector alone gave no colonies.

GDC-2 protein from pAX624-containing cells was isolated by expression of GDC-2-6×His-tagged protein in E. coli, and the resulting protein purified using Ni-NTA Superflow Resin (Qiagen) as per manufacturer's instructions.

### EXAMPLE 10

### Assay of GDC-2 Acetolactate Synthase Activity

Acetolactate synthases are decarboxylating enzymes that condense two pyruvate molecules to form acetolactate with the release of a CO₂ moiety from one of the pyruvate substrates. In the detection of the enzymatic reaction described by Pang and Duggleby (Pang and Duggleby (1999) Biochemistry 18:5222-5231), the product acetolactate is converted to acetoin by incubation with 1% H₂SO₄ for 15 minutes at 60° C. followed by neutralization with KOH. The acetoin is then detected as described by Westerfeld (Westerfeld (1945) J. Biol. Chem. 161:495-502), using 0.15% creatine and 1.5% alpha-naphthol (dissolved in 2.5 N NaOH). The red colored reaction product is quantified by absorbance at 525 nm.

Samples containing either 5 μg or 10 μg of GDC-2 were incubated in 50 mM pyruvate, 1 mM thymine pyrophosphate, 10 mM MgCl₂, 0.01 mM Flavin adenine dinucleotide (FAD), 100 mM potassium phosphate buffer pH 7.0 (total reaction volume of 50 μl) for 2 hours at 37° C. The reaction was stopped by the addition of 1 μl of 50% sulfuric acid (H₂SO₄) and incubated at 60° C. for 15 minutes. The reaction was neutralized by the addition of 30 μl of 1 N KOH followed by the addition of 10 μl of 1.5% creatine and 10 μl of 15% alpha-napthol dissolved in 2.5 N NaOH. The red colored reaction product was quantified by absorbance at 525 nm.

TABLE 4
Acetolactate synthase activity

| Amount GDC-2 (μg) | Absorbance 525 nm) |
|---|---|
| 0 μg (control) | 0.0 |
| 5 μg | 1.99 |
| 10 μg | 3.13 |

### EXAMPLE 11

### Engineering GDC-1 for Plant Transformation

The GDC-1 open reading frame (ORF) was amplified by PCR from a full-length cDNA template. HindIII restriction sites were added to each end of the ORF during PCR. Additionally, the nucleotide sequence ACC was added immediately 5′ to the start codon of the gene to increase translational efficiency (Kozak (1987) Nucleic Acids Research 15:8125-8148; and Joshi (1987) Nucleic Acids Research 15:6643-6653). The PCR product was cloned and sequenced, using techniques well known in the art, to ensure that no mutations were introduced during PCR.

The plasmid containing the GDC-1 PCR product was partially digested with Hind III and the 1.7 kb Hind III fragment containing the intact ORF was isolated. (GDC-1 contains an internal Hind III site in addition to the sites added by PCR.) This fragment was cloned into the Hind III site of plasmid pAX200, a plant expression vector containing the rice actin promoter (McElroy et al. (1991) Molecular General Genetics 231:150-160) and the PinII terminator (An et al. (1989) The Plant Cell 1:115-122). The promoter—gene—terminator fragment from this intermediate plasmid was subcloned into Xho I site of plasmid pSB11 (Japan Tobacco, Inc.) to form the plasmid pAX810. pAX810 is organized such that the 3.45 kb DNA fragment containing the promoter—GDC-1—terminator construct may be excised from pAX810 by double digestion with KpnI and XbaI for transformation into plants using aerosol beam injection. The structure of pAX810 was verified by restriction digests and gel electrophoresis and by sequencing across the various cloning junctions.

Plasmid pAX810 was mobilized into Agrobacterium tumifaciens strain LBA4404 which also harbored the plasmid pSB1 (Japan Tobacco, Inc.), using triparental mating procedures well known in the art, and plating on media containing spectinomycin. Plasmid pAX810 carries spectinomycin resistance but is a narrow host range plasmid and cannot replicate in Agrobacterium. Spectinomycin resistant colonies arise when pAX810 integrates into the broad host range plasmid pSB1 through homologous recombination. The cointegrate product of pSB1 and pAX810 recombination (pAX204) was verified by Southern hybridization (data not shown). The Agrobacterium strain harboring pAX204 was used to transform maize by the PureIntro method (Japan Tobacco).

### EXAMPLE 12

### Engineering GDC-2 for Plant Transformation

The GDC-2 open reading frame (ORF) was amplified by polymerase chain reactions from a full-length cDNA template. Hind III restriction sites were added to each end of the ORF during PCR. Additionally, the nucleotide sequence ACC was added immediately 5′ to the start codon of the gene to increase translational efficiency (Kozak (1987) 15:8125-8148; Joshi (1987) Nucleic Acids Research 15:6643-6653). The PCR product was cloned and sequenced, using techniques well known in the art, to ensure that no mutations were introduced during PCR.

The plasmid containing the GDC-2 PCR product was digested with Hind III and the fragment containing the intact ORF was isolated. This fragment was cloned into the Hind III site of plasmid pAX200, a plant expression vector containing the Rice Actin promoter (McElroy et al. (1991) Molecular General Genetics 231:150-160) and the PinII terminator (An et al. (1989) The Plant Cell 1:115-122). PAX811 is organized such that the 3.91 kb DNA fragment containing the promoter—GDC-2—terminator construct may be excised from pAX811 by double digestion with Kpn I and Pme I and used for transformation into plants by aerosol beam injection. The structure of pAX811 was verified by restriction digests and gel electrophoresis and by sequencing across the various cloning junctions.

Plasmid pAX810 was mobilized into Agrobacterium tumifaciens strain LBA4404 which also harbored the plasmid pSB1 (Japan Tobacco, Inc.), using triparental mating procedures well known in the art, and plating on media containing spectinomycin. Plasmid pAX811 carries spectinomycin resistance but is a narrow host range plasmid and cannot replicate in Agrobacterium. Spectinomycin resistant colonies arise when pAX811 integrates into the broad host range plasmid pSB1 through homologous recombination. The cointegrate product of pSB1 and pAX811 recombination (pAX205) was verified by Southern hybridization (data not shown). The Agrobacterium strain harboring pAX205 was used to transform maize by the PureIntro method (Japan Tobacco).

### EXAMPLE 13

### Transformation of GDC-1 and GDC-2 into Plant Cells

Maize ears are collected 8-12 days after pollination. Embryos are isolated from the ears, and those embryos 0.8-1.5 mm in size are used for transformation. Embryos are plated scutellum side-up on a suitable incubation media, such as DN62A5S media (3.98 g/L N6 Salts; 1 mL/L (of 1000× Stock) N6 Vitamins; 800 mg/L L-Asparagine; 100 mg/L Myo-inositol; 1.4 g/L L-Proline; 100 mg/L Casaminoacids; 50 g/L sucrose; 1 mL/L (of 1 mg/mL Stock) 2,4-D). However, media and salts other than DN62A5S are suitable and are known in the art. Embryos are incubated overnight at 25° C. in the dark.

The resulting explants are transferred to mesh squares (30-40 per plate), transferred onto osmotic media for 30-45 minutes, then transferred to a beaming plate (see, for example, PCT Publication No. WO/0138514 and U.S. Pat. No. 5,240,842).

DNA constructs designed to express GDC-1, GDC-2 or GDC-1 and GDC-2 in plant cells are accelerated into plant tissue using an aerosol beam accelerator, using conditions essentially as described in PCT Publication No. WO/0138514. After beaming, embryos are incubated for 30 min on osmotic media, and placed onto incubation media overnight at 25° C. in the dark. To avoid unduly damaging beamed explants, they are incubated for at least 24 hours prior to transfer to recovery media. Embryos are then spread onto recovery period media, for 5 days, 25° C. in the dark, then transferred to a selection media. Explants are incubated in selection media for up to eight weeks, depending on the nature and characteristics of the particular selection utilized. After the selection period, the resulting callus is transferred to embryo maturation media, until the formation of mature somatic embryos is observed. The resulting mature somatic embryos are then placed under low light, and the process of regeneration is initiated by methods known in the art. The resulting shoots are allowed to root on rooting media, and the resulting plants are transferred to nursery pots and propagated as transgenic plants.

Materials

DN62A5S MEDIA

| Components | per liter | | Source |
|---|---|---|---|
| Chu'S N6 Basal Salt | 3.98 | g/L | Phytotechnology Labs |
| Mixture (Prod. | | | |
| No. C 416) | | | |
| Chu's N6 Vitamin | 1 | mL/L | Phytotechnology Labs |
| Solution | | (of 1000x Stock) | |
| (Prod. No. C 149) | | | |
| L-Asparagine | 800 | mg/L | Phytotechnology Labs |
| Myo-inositol | 100 | mg/L | Sigma |
| L-Proline | 1.4 | g/L | Phytotechnology Labs |
| Casaminoacids | 100 | mg/L | Fisher Scientific |
| Sucrose | 50 | g/L | Phytotechnology Labs |
| 2,4-D (Prod. No. | 1 | mL/L | Sigma |
| D-7299) | | (of 1 mg/mL Stock) | |

Adjust the pH of the solution to pH to 5.8 with 1N KOH/1N KCl, add Gelrite (Sigma) to 3 g/L, and autoclave. After cooling to 50° C., add 2 ml/L of a 5 mg/ml stock solution of Silver Nitrate (Phytotechnology Labs). Recipe yields about 20 plates.

### EXAMPLE 14

### Transformation of GDC-1 and GDC-2 into Plant Cells by Agrobacterium-Mediated Transformation

Ears are collected 8-12 days after pollination. Embryos are isolated from the ears, and those embryos 0.8-1.5 mm in size are used for transformation. Embryos are plated scutellum side-up on a suitable incubation media, and incubated overnight at 25° C. in the dark. However, it is not necessary per se to incubate the embryos overnight. Embryos are contacted with an Agrobacterium strain containing the appropriate vectors for Ti plasmid mediated transfer for 5-10 min, and then plated onto co-cultivation media for 3 days (25° C. in the dark). After co-cultivation, explants are transferred to recovery period media for five days (at 25° C. in the dark). Explants are incubated in selection media for up to eight weeks, depending on the nature and characteristics of the particular selection utilized. After the selection period, the resulting callus is transferred to embryo maturation media, until the formation of mature somatic embryos is observed. The resulting mature somatic embryos are then placed under low light, and the process of regeneration is initiated as known in the art. The resulting shoots are allowed to root on rooting media, and the resulting plants are transferred to nursery pots and propagated as transgenic plants.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A method for conferring resistance to an herbicide in a cell, comprising transforming said cell with a DNA construct, said construct comprising a promoter that drives expression in said cell, operably linked to a nucleotide sequence encoding a decarboxylase, wherein expression of said decarboxylase in said cell confers resistance to at least one herbicide.

2. The method of claim 1, wherein said decarboxylase is selected from the group consisting of a pyruvate decarboxylase, a benzoylformate decarboxylase, an oxalyl-CoA decarboxylase, a 2-oxoglutarate decarboxylase, an indolepyruvate decarboxylase, a 5-guanidino-2-oxopentanoate decarboxylase, a phenylglyoxylate dehydrogenase (acylating), a pyruvate dehydrogenase (cytochrome), a pyruvate oxidase, a pyruvate dehydrogenase (lipoamide), an oxoglutarate dehydrogenase (lipoamide), a transketolase, a formaldehyde transketolase, an acetoin-ribose-5-phosphate transaldolase, a tartronate-semialdehyde synthase, a phosphoketolase, a fructose-6-phosphate phosphoketolase, a benzoin aldolase, a 2-hydroxy-3-oxoadipate synthase, an acetolactate synthase, an 1-deoxy-C-xylulose 5-phosphate synthase, and a sulfoacetaldehyde lyase.

3. The method of claim 1, wherein said nucleotide sequence has at least 90% identity with the nucleotide sequence of SEQ ID NO:1, 2, 13, 14, 21, or 23.

4. The method of claim 1, wherein expression of the protein results in increased tolerance of the cell to more than one herbicide.

5. The method of claim 1, wherein said herbicide is a glyphosate.

6. The method of claim 1, wherein said decarboxylase has a TPP-binding domain.

7. The method of claim 1, wherein said cell is a plant cell.

8. The method of claim 1, wherein said cell is a bacterial cell.

9. A transformed plant cell, said cell comprising a DNA construct, said construct comprising a promoter that drives expression in a plant cell operably linked with a nucleotide sequence that encodes a decarboxylase, wherein expression of said decarboxylase in said cell confers resistance to at least one herbicide.

10. The plant cell of claim 9, wherein said nucleotide sequence has at least 90% identity with the nucleotide sequence of SEQ ID NO:1, 2, 13, 14, 21, or 23.

11. A plant regenerated from the plant cell of claim 9, wherein said plant is resistant to at least one herbicide.

12. Transformed seed of a plant of claim 11.

13. A plant having stably incorporated into its genome a DNA construct comprising a promoter that drives expression in a plant cell operably linked with a nucleotide sequence that encodes a decarboxylase, wherein expression of said decarboxylase in said cell confers resistance to at least one herbicide.

14. The plant of claim 13, wherein said nucleotide sequence has at least 90% identity with the nucleotide sequence of SEQ ID NO:1, 2, 13, 14, 21, or 23.

15. The plant of claim 13, wherein said plant is selected from the group consisting of maize, sorghum, wheat, sunflower, tomato, crucifers, peppers, potato, cotton, rice, soybean, sugarbeet, sugarcane, tobacco, barley, and oilseed rape.

16. A transformed bacterial cell, said cell comprising a DNA construct, said construct comprising a promoter that drives expression in a bacterial cell operably linked with a nucleotide sequence that encodes a decarboxylase, wherein expression of said decarboxylase in said cell confers resistance to at least one herbicide.

17. The bacterial cell of claim 16, wherein said nucleotide sequence has at least 90% identity with the nucleotide sequence of SEQ ID NO:1, 2, 13, 14, 21, or 23.
